(19) United States
(12) Patent Application Publication
(10) Pub. No.: US 2004/0082563 A1
(43) Pub. Date: Apr. 29, 2004

(54) Phenyl derivatives 3

(76) Inventors: Dieter Dorsch, Ober-Ramstadt (DE); Bertram Cezanne, Morfelden-Walldorf (DE); Werner Mederski, Erzhausen (DE); Christos Tsaklakidis, Weinheim (DE); Johannes Gleitz, Darmstadt (DE); Christopher Barnes, Bad Soden (DE)

Correspondence Address: Millen White, Zelano & Branigan, Arlington Courthouse Plaza I, 2200 Clarendon Boulevard Suite 1400, Arlington, VA, 22201, US

(21) Appl. No.: 10/471,768

(22) PCT Filed: Feb. 27, 2002
(86) PCT No.: PCT/EP02/02092

(30) Foreign Application Priority Data
Mar. 16, 2001 (DE) 101 12 768.5

(51) Int. Cl. C07D 417/02; C07D 413/02; C07D 271/06; C07D 271/12

(52) U.S. Cl. 514/210.2; 514/217.1; 514/227.5; 514/227.8; 514/238.5; 514/254.04; 514/255.03; 514/326; 514/364; 514/408; 540/603; 544/60; 544/138; 544/162; 544/367; 544/392; 546/209; 546/229; 548/132; 548/131; 548/950

(57) ABSTRACT

Novel compounds of the formula (I) formula (I) in which W, E, X, Y, T, R1, R², R^{2′}, and R^{2″} are as defined in Patent claim 1, are inhibitors of coagulation factor Xa and can be employed for the prophylaxis and/or therapy of thromboembolic disorders and for the treatment of tumours.

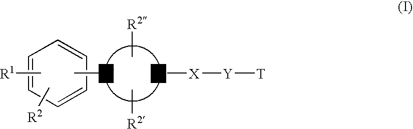

The invention relates to compounds of the formula I

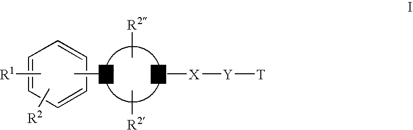

in which

R¹ is H, CN, or is —C(═NH)—NH₂, CON(R³)₂ or —[C(R⁴)₂]ₙN(R³)₂, each of which is unsubstituted or monosubstituted by C(═O)R³, COOR³, OR³ or by a conventional amino-protecting group, or is

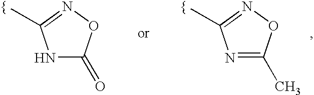

R², R^{2′}

and R are each, independently of one another, H, Hal, A, OR³, N(R³)₂, NO₂, CN, —[C(R⁴)₂]ₙ—Ar, —[C(R⁴)₂]ₙ-Het, —[C(R⁴)₂]ₙ-cycloalkyl, ═C(R⁴)—[C(R⁴)₂]ₙ—COOR³═C(R⁴)—[C(R⁴)₂]ₙ—CON(R³)₂, —[C(R⁴)₂]ₙ—COOR³, —[C(R⁴)₂]ₙ—CON(R³)₂, O—[C(R⁴)₂]ₙ—COOR³

or O—[C(R⁴)₂]ₙ—CON(R³)₂,

R³ is H, A, —[C(R⁴)₂]ₙ—Ar, —[C(R⁴)₂]ₙ-Het or —[C(R⁴)₂]ₙ-cycloalkyl,

R⁴ is H or A,

W is N, CR³ or an sp²-hybridised carbon atom,

E together with W is a 3- to 7-membered saturated carbocyclic or heterocyclic ring having from 0 to 3 N atoms, from 0 to 2 O atoms and/or from 0 to 2 S atoms, which

a) may contain a double bond

to which

b) may be fused a benzene ring or a saturated, unsaturated or aromatic heterocyclic ring,

which

c) may be substituted by carbonyl oxygen and/or by R² and/or R²,

X is —[C(R⁴)₂]ₙCON R³[C(R⁴)₂]ₙ—, —[C(R⁴)₂]ₙN R³CO[C(R⁴)₂]ₙ—, —[C(R⁴)₂]ₙNR³[C(R⁴)₂]ₙ—or —[C(R⁴)₂]ₙO[C(R⁴)₂]ₙ—,

Y is alkylene, cycloalkylene, Het-diyl or Ar-diyl,

T is a monocyclic or bicyclic, saturated or unsaturated heterocyclic ring having from 1 to 4 N, O and/or S atoms, which is monosubstituted or disubstituted by carbonyl oxygen and which may additionally be monosubstituted, disubstituted or trisubstituted by Hal, A, —[C(R⁴)₂]ₙ—Ar, —[C(R⁴)₂]ₙ-Het, —[C(R⁴)₂]ₙ-cycloalkyl, OR³, N(R³)₂, NO₂, CN, COOR³, CON(R³)₂, NR³COA, NR₃CON(R₃)₂, NR³SO₂A, COR³, SO₂NR³ or S(O)ₘA,

A is unbranched or branched alkyl having 1-6 carbon atoms, in which one or two CH₂ groups may be replaced by O or S atoms and/or by —CH═CH— groups and/or, in addition, 1-7H atoms may be replaced by F,

Ar is phenyl, naphthyl or biphenyl, each of which is unsubstituted or monosubstituted, disubstituted or trisubstituted by Hal, A, OR⁴, N(R⁴)₂, NR⁴CON(R⁴)₂, NO₂, CN, COOR⁴, CON(R⁴)₂, NR⁴COA, NR⁴SO₂A, COR⁴, SO₂NR⁴ or S(O)ₘA,

Het is a monocyclic or bicyclic, saturated, unsaturated or aromatic heterocyclic ring having from 1 to 4 N, O and/or S atoms which is unsubstituted or monosubstituted, disubstituted or trisubstituted by Hal, A, —[C(R⁴)₂]ₙ—Ar, —[C(R⁴)₂]ₙ-Het′, [C(R⁴)₂]ₙ-cycloalkyl, —[C(R⁴)₂]ₙ—CON(R³)₂, —[C(R⁴)₂]ₙ—COOR³, OR³, N(R³)₂, NR³CON(R³)₂, NO₂, CN,

NR³COA, NR³SO₂A, COR³, SO₂NR³, S(O)ₘA and/or carbonyl oxygen,

Het′ is a monocyclic or bicyclic, saturated, unsaturated or aromatic heterocyclic ring having from 1 to 4 N, O and/or S atoms which is unsubstituted or monosubstituted or disubstituted by Hal, A, OR³, N(R³)₂, NO₂, CN, COOR³, CON(R³)₂, NR³COA, NR³SO₂A, COR³, SO₂NR³, S(O)ₘA and/or carbonyl oxygen,

Hal is F, Cl, Br or I,

m and n are each, independently of one another, 0, 1 or 2,

and pharmaceutically usable derivatives, solvates and stereoisomers thereof, including mixtures thereof in all ratios.

The invention had the object of finding novel compounds having valuable properties, in particular those which can be used for the preparation of medicaments.

It has been found that the compounds of the formula I and their salts have very valuable pharmacological properties and are well tolerated. In particular, they exhibit factor Xa-inhibiting properties and can therefore be employed for combating and preventing thromboembolic disorders, such as thrombosis, myocardial infarction, arteriosclerosis, inflammation, apoplexia, angina pectoris, restenosis after angioplasty and claudicatio intermittens.

The compounds of the formula I according to the invention may furthermore be inhibitors of the coagulation factors factor Vila, factor IXa and thrombin in the blood coagulation cascade.

Aromatic amidine derivatives having an antithrombotic action are disclosed, for example, in EP 0 540 051 B1, WO 98/28269, WO 00/71508, WO 00/71511, WO 00/71493, WO 00/71507, WO 00/71509, WO 00/71512, WO 00/71515 and WO 00/71516. Cyclic guanidines for the treatment of thromboembolic disorders are described, for example, in WO 97/08165. Aromatic heterocyclic compounds having a factor Xa inhibitory activity are disclosed, for example, in WO 96/10022. Substituted N-[(aminoiminomethyl)phenylalkyl]azaheterocyclylamides as factor Xa inhibitors are described in WO 96/40679.

The antithrombotic and anticoagulant effect of the compounds according to the invention is attributed to the inhibitory action against activated coagulation protease, known by the name factor Xa, or to the inhibition of other activated serine proteases, such as factor VIIa, factor IXa or thrombin.

Factor Xa is one of the proteases involved in the complex process of blood coagulation. Factor Xa catalyses the conversion of prothrombin into thrombin. Thrombin cleaves fibrinogen into fibrin monomers, which, after crosslinking, make an elementary contribution to thrombus formation. Activation of thrombin may result in the occurrence of thromboembolic disorders. However, inhibition of thrombin may inhibit the fibrin formation involved in thrombus formation.

The inhibition of thrombin can be measured, for example by the method of G. F. Cousins et al. in Circulation 1996, 94, 1705-1712.

Inhibition of factor Xa can thus prevent the formation of thrombin.

The compounds of the formula I according to the invention and their salts engage in the blood coagulation process by inhibiting factor Xa and thus inhibit the formation of thrombuses.

The inhibition of factor Xa by the compounds according to the invention and the measurement of the anticoagulant and antithrombotic activity can be determined by conventional in-vitro or in-vivo methods. A suitable method is described, for example, by J. Hauptmann et al. in Thrombosis and Haemostasis 1990, 63, 220-223.

The inhibition of factor Xa can be measured, for example by the method of T. Hara et al. in Thromb. Haemostas. 1994, 71, 314-319.

Coagulation factor VIIa initiates the extrinsic part of the coagulation cascade after binding to tissue factor and contributes to the activation of factor X to give factor Xa. Inhibition of factor VIIa thus prevents the formation of factor Xa and thus subsequent thrombin formation.

The inhibition of factor VIIa by the compounds according to the invention and the measurement of the anticoagulant and antithrombotic activity can be determined by conventional in-vitro or in-vivo methods. A conventional method for the measurement of the inhibition of factor VIIa is described, for example, by H. F. Ronning et al. in Thrombosis Research 1996, 84, 73-81.

Coagulation factor IXa is generated in the intrinsic coagulation cascade and is likewise involved in the activation of factor X to give factor Xa. Inhibition of factor IXa can therefore prevent the formation of factor Xa in a different way.

The inhibition of factor IXa by the compounds according to the invention and the measurement of the anticoagulant and antithrombotic activity can be determined by conventional in-vitro or in-vivo methods. A suitable method is described, for example, by J. Chang et al. in Journal of Biological Chemistry 1998, 273, 12089-12094.

The compounds according to the invention may furthermore be used for the treatment of tumours, tumour illnesses and/or tumour metastases.

A correlation between tissue factor TF/factor Vila and the development of various types of cancer has been indicated by T. Taniguchi and N. R. Lemoine in Biomed. Health Res. (2000), 41 (Molecular Pathogenesis of Pancreatic Cancer), 57-59.

The publications listed below describe an antitumoural action of TF-VII and factor Xa inhibitors for various types of tumour:

K. M. Donnelly et al. in Thromb. Haemost. 1998; 79: 1041-1047;

E. G. Fischer et al. in J. Clin. Invest. 104: 1213-1221 (1999);

B. M. Mueller et al. in J. Clin. Invest. 101: 1372-1378 (1998);

M. E. Bromberg et al. in Thromb. Haemost. 1999; 82: 88-92

The compounds of the formula I can be employed as medicament active ingredients in human and veterinary medicine, in particular for the treatment and prevention of thromboembolic disorders, such as thrombosis, myocardial infarction, arteriosclerosis, inflammation, apoplexia, angina pectoris, restenosis after angioplasty, claudicatio intermiftens, venous thrombosis, pulmonary embolism, arterial thrombosis, myocardial ischaemia, unstable angina and strokes based on thrombosis.

The compounds according to the invention are also employed for the treatment or prophylaxis of atherosclerotic diseases, such as coronary arterial disease, cerebral arterial disease or peripheral arterial disease.

The compounds are also employed in combination with other thrombolytic agents in myocardial infarction, furthermore for prophylaxis for reocclusion after thrombolysis, percutaneous transluminal angioplasty (PTCA) and coronary bypass operations.

The compounds according to the invention are furthermore used for the prevention of rethrombosis in microsurgery, furthermore as anticoagulants in connection with artificial organs or in haemodialysis.

The compounds are furthermore used in the cleaning of catheters and medical aids in patients in vivo, or as anticoagulants for the preservation of blood, plasma and other blood products in vitro. The compounds according to the invention are furthermore used for diseases in which blood coagulation makes a crucial contribution toward the course of the disease or represents a source of secondary pathology, such as, for example, in cancer, including metastasis, inflammatory disorders, including arthritis, and diabetes.

In the treatment of the disorders described, the compounds according to the invention are also used in combination with other thrombolytically active compounds, such as, for example, with the “tissue plasminogen activator” t-PA, modified t-PA, streptokinase or urokinase. The compounds according to the invention are administered either at the same time as or before or after the other substances mentioned.

Particular preference is given to simultaneous administration with aspirin in order to prevent recurrence of the clot formation.

The compounds according to the invention are also used in combination with blood platelet glycoprotein receptor (IIb/IIIa) antagonists, which inhibit blood platelet aggregation.

The invention relates to the compounds of the formula I and their salts and to a process for the preparation of compounds of the formula I according to claim 1 and their salts, characterised in that

a) they are liberated from one of their functional derivatives by treatment with a solvolysing or hydrogenolysing agent by

i) liberating an amidino group from their hydroxyl, oxadiazole or oxazolidinone derivative by hydrogenolysis or solvolysis,

ii) replacing a conventional amino-protecting group with hydrogen by treatment with a solvolysing or hydrogenolysing agent, or liberating an amino group protected by a conventional protecting group, or

b) a cyano group is converted into an N-hydroxyamidino group, or

c) for the preparation of a compound of the formula I in which X is —[C(R⁴)₂]ₙCONR³[C(R⁴)₂]ₙ—, —[C(R⁴)₂]ₙNR³[C(R⁴)₂]ₙ—or —[C(R⁴)₂]ₙO[C(R⁴)₂]ₙ—,

a compound of the formula II

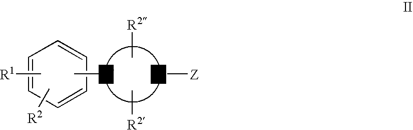

in which

Z is —[C(R⁴)₂]ₙCO-L or —[C(R⁴)₂]ₙ-L,

L is Cl, Br, I or a free or reactively functionally modified OH group, and

R¹, R², R², R^{2″}, R⁴, n, W and E are as defined in claim 1,

with the proviso that any free amino group present is protected,

is reacted with a compound of the formula III

Q-Y-T III

in which

Q is HNR³[C(R⁴)₂]ₙ-Y-T or HO[C(R⁴)₂]ₙ-Y-T,

and R³, R⁴, n, Y and T are as defined in claim 1,

and, where appropriate, a protecting group is subsequently removed or

d) for the preparation of a compound of the formula I

in which X is —[C(R⁴)₂]ₙNR³CO[C(R⁴)₂]ₙ—,

a compound of the formula IV

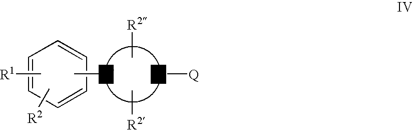

in which

Q is —[C(R⁴)₂]ₙNHR³,

and R¹, R², R^{2′}, R^{2″}, R³, R⁴, n, W und E are as defined in claim 1,

with the proviso that any further free amino group present is protected,

is reacted with a compound of the formula V

Z-Y-T V

in which

Z is L-C(═O)—[C(R⁴)₂]ₙ-Y-T, and

L is Cl, Br, I or a free or reactively functionally modified OH group, and

n, Y and T are as defined in claim 1,

and, where appropriate, a protecting group is subsequently removed, and/or

e) a base or acid of the formula I is converted into one of its salts.

The invention also relates to the optically active forms (stereoisomers), the enantiomers, the racemates, the diastereomers and the hydrates and solvates of these compounds. The term solvates of the compounds is taken to mean adductions of inert solvent molecules onto the compounds which form owing to their mutual attractive force. Solvates are, for example, mono- or dihydrates or alcoholates.

The term “pharmaceutically usable derivatives” is taken to mean, for example, the salts of the compounds according to the invention and so-called prodrug compounds.

The term “prodrug derivatives” is taken to mean compounds of the formula I which have been modified with, for example, alkyl or acyl groups, sugars or oligopeptides and which are rapidly cleaved in the organism to form the active compounds according to the invention.

These also include biodegradable polymer derivatives of the compounds according to the invention, as described, for example, in Int. J. Pharm. 115, 61-67 (1995).

The invention also relates to mixtures of the compounds of the formula I according to the invention, for example mixtures of two diasteriomers, for example in the ratio 01:01, 01:02, 01:03, 01:04, 01:05, 01:10, 1:100 or 1:1000.

These are particularly preferably mixtures of stereoisomeric compounds.

In particular, the invention also relates to the —C(═NH)—NH₂ compounds of the formula I which are substituted by —COA, —COOA, —OH or by a conventional amino-protecting group.

For all radicals which occur more than once, such as, for example, A, their meanings are independent of one another.

Above and below, the radicals and parameters W, E, X, Y, T, R¹, R², R^{2′} and R^{2″} are as defined under the formula 1, unless expressly stated otherwise.

A is alkyl, is unbranched (linear) or branched, and has 1, 2, 3, 4, 5, 6, 7, 8, 9 or 10 carbon atoms. A is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

A is very particularly preferably alkyl having 1-6 carbon atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl.

Cycloalkyl is preferably cyclopropyl, cyclobutyl, cyclopentyl, cyclohexyl or cycloheptyl.

Alkylene is preferably methylene, ethylene, propylene, butylene, pentylene or hexylene, furthermore branched alkylene.

—COR³ (acyl) is preferably formyl, acetyl, propionyl, furthermore also butyryl, pentanoyl, hexanoyl or, for example, benzoyl.

Ph is phenyl, Me is methyl, Et is ethyl, BOC is tert-butoxycarbonyl.

Hal is preferably F, Cl or Br, but alternatively I.

If R¹ is CON(R³)₂ or —[C(R⁴)₂]ₙN(R³)₂, CONH₂, NH₂ or CH₂NH₂ is preferred.

R¹ is particularly preferably CN, NH₂, CH₂NH₂, CH₂CH₂NH₂, CONH₂, —C(—NH)—NH₂ which is unsubstituted or monosubstituted by OH,

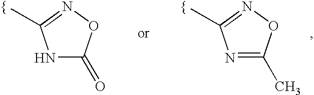

R² is preferably H.

R³ is preferably H, A or —(CH₂)ᵣ—Ar, particularly preferably, for example, H, alkyl having 1-6 carbon atoms, phenyl or benzyl.

X is preferably, for example, CONR³, CH₂CONR³, CH₂NR³, CONR³CH₂, CH₂O, CH₂OCH₂ or OCH₂,

where R is hydrogen, alkyl having 1, 2, 3, 4, 5 or 6 carbon atoms, Phenyl or benzyl.

X is very particularly preferably CONH, CONHCH₂, CH₂NH or CH₂O.

Y is preferably alkylene or Ar-diyl, particularly preferably methylene, ethylene, propylene, or 1,4-phenylene which is unsubstituted or monosubstituted by F, ethoxycarbonylmethoxy or carboxymethoxy, furthermore alternatively pyridinedyl, preferably pyridine-2,5-diyl. Y is in particular 1,3- or 1,4-phenylene. T is preferably a monocyclic or bicyclic, saturated or unsaturated heterocyclic radical having 1 or 2 N or O atoms which is monosubstituted or disubstituted by carbonyl oxygen. T is particularly preferably, for example, 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2,6-dioxopiperidin 1-yl, 2-oxopiperazin-1-yl, 2,5-dioxopyrrolidin-1-yl, 2-oxo-1,3-oxazolidin-3-yl, 3-oxo-2H-pyridazin-2-yl or 2-oxoazepan-1-yl.

Ar is preferably unsubstituted phenyl, naphthyl or biphenyl, furthermore preferably phenyl, naphthyl or biphenyl, each of which is monosubstituted, disubstituted or trisubstituted by A, fluorine, chlorine, bromine, iodine, hydroxyl, methoxy, ethoxy, propoxy, butoxy, pentyloxy, hexyloxy, nitro, cyano, formyl, acetyl, propionyl, trifluormethyl, amino, methylamino, ethylamino, dimethylamino, diethylamino, benzyloxy, sulfonamido, methylsulfonamido, ethylsulfonamido, propylsulfonamido, butylsulfonamido, dimethylsulfonamido, phenylsulfonamido, carboxy, methoxycarbonyl, ethoxycarbonyl or aminocarbonyl.

Ar is particularly preferably, for example, phenyl which is unsubstituted or monosubstituted or disubstituted by Hal, A, OH or methoxy substituiertes phenyl.

Het is preferably, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, I— or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8 isoquinolyl, 3-, 4-, 5-, 6-, 7- or 8-quinolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxan-6-yl, 2,1,3-benzothiadiazol-4- or -5-yl or 2,1,3-benzoxadiazol-5-yl.

The heterocyclic radicals may also be partially or fully hydrogenated.

Het can thus also be, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, 2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4 or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, 6-, -7- or -8-quiinolyl, 1,2,3,4-tetrahydro-1-, -2-, -3-, -4-, -5-, -6-, -7- or -8 isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or alternatively 3,4-dihydro-2H-1,5-benzodioxepin-6- or -7-yl, furthermore preferably 2,3-dihydrobenzofuranyl or 2,3-dihydro-2-oxofuranyl.

Het is very particularly preferably a monocyclic or bicyclic, saturated or unsaturated heterocyclic radical having 1 or 2 N or O atoms which is unsubstituted or monosubstituted or disubstituted by carbonyl oxygen, such as, for example, morpholin-4-yl, 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2,6-dioxopiperidin1-yl, 2-oxopiperazin-1-yl, 2,5-dioxopyrrolidin-1-yl, 2-oxo-1,3-oxazolidin-3-yl, 3-oxo-2H-pyridazin-2-yl, 2-Azabicyclo[2.2.2]octan-3-on-2-yl or 2-caprolactam-1 yl.

Het′ is preferably, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8 isoquinolyl, 3-, 4-, 5-, 6-, 7- or 8-quinolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxan-6-yl, 2,1,3-benzothiadiazol-4- or -5-yl or 2,1,3-benzoxadiazol-5-yl.

The heterocyclic radicals may also be partially or fully hydrogenated. Het′ can thus also be, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4-or -5-pyrrolyl, 1-, 2- or 3-pyrrolidinyl, tetrahydro-1-2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4 or -5-yl, hexahydro-1-, -3- or -4-pyridazinyl, hexahydro-1-, -2-, -4-or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8 isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylendioxyphenyl, 3,4-(difluoromethylenedioxy)phenyl, 2,3-dihydrobenzofuran-5- or 6-yl, 2,3-(2-oxomethylenedioxy)phenyl or alternatively 3,4-dihydro-2H-1,5-benzodioxepin-6- or -7-yl, furthermore preferably 2,3-dihydrobenzofuranyl or 2,3-dihydro-2-oxofuranyl.

m is preferably 2, furthermore also 0 or 1.

n is preferably 1, furthermore also 0 or 2.

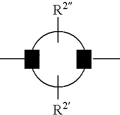

is preferably

a 3- to 7-membered saturated carbocyclic or heterocyclic ring having from 0 to 2 N atoms which

a) may contain a double bond

to which

b) a benzene ring or a 5- to 6-membered aromatic heterocyclic ring having 1-2 N atoms may be fused,

where

R²′ is particularly preferably H, Hal, A, ═CH—COOA, ═CH—CONH₂ or O—CH₂—COOH, and

R^{2″} is, in particular, H.

The aromatic heterocyclic ring mentioned under b) is preferably imidazole or pyridine.

The compounds of the formula I may have one or more chiral centres and therefore occur in various stereoisomeric forms. The formula I covers all these forms.

Accordingly, the invention relates in particular to the compounds of the formula I in which at least one of the said radicals has one of the preferred meanings indicated above. Some preferred groups of compounds may be expressed by the following sub-formulae Ia to Iw, which conform to the formula I and in which the radicals not designated in greater detail are as defined under the formula 1, but in which

in Ia R² is H;

in Ib R¹ is —C(═NH)—NH₂, which is unsubstituted or monosubstituted by OH, or is

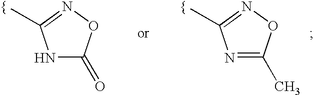

in Ic R¹ is CONH₂, CH₂NH₂, or —C(═NH)—NH₂, which is unsubstituted or monosubstituted by OH, and

R² is H;

in Id R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH, and

R^{2″} is H;

in Ie R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H, and

R³ is H, A or —(CH₂)ₙ—Ar;

in If R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H, and

R³ is H, alkyl having 1-6 carbon atoms, phenyl or benzyl;

in Ig Ar is phenyl, which is unsubstituted or monosubstituted or disubstituted by Hal, OR⁴, SO₂NH₂, SO₂A or NHCONH₂;

in Ih X is CONR³, CH₂CONR³, CH₂NR³, CONR³CH₂, CH₂O, CH₂OCH₂ or OCH₂,

R³ is H, alkyl having 1, 2, 3, 4, 5 or 6 carbon atoms, phenyl or benzyl;

in Ii R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

X is CONR³, CH₂CONR³, CH₂NR³, CONR³CH₂, CH₂O, CH₂OCH₂ or OCH₂,

R³ is H, alkyl having 1, 2, 3, 4, 5 or 6 carbon atoms, phenyl or benzyl;

in Ij R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

X is CONH, CONHCH₂, CH₂NH or CH₂O,

R³ is H, alkyl having 1, 2, 3, 4, 5 or 6 carbon atoms, phenyl or benzyl;

in Ik W is N or CH or an sp²-hybridised carbon atom,

E together with W is a 3- to 7-membered saturated carbocyclic or heterocyclic ring having from 0 to 2 N atoms

which

a) may contain a double bond

and to which

b) may be fused a benzene ring or a saturated or aromatic heterocyclic ring having 1-2 N atoms;

in Il Y is Ar-diyl;

in Im Y is Ar-diyl, and

Ar is phenyl, which is unsubstituted or monosubstituted or disubstituted by Hal, OR⁴, SO₂NH₂, SO₂A or NHCONH₂;

in In Y is 1,4-phenylene;

in Io T is a monocyclic or bicyclic, saturated or unsaturated heterocyclic ring having 1 or 2 N and/or O atoms, which is monosubstituted or disubstituted by carbonyl oxygen;

in Ip R¹ is CONH₂, CH₂NH₂, or —C(═NH)—NH₂, which is unsubstituted or monosubstituted by OH,

R² is H,

R^{2′} is H, Hal, A, —CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH pr an sp²-hybridised carbon atom,

E together with W is a 3- to 7-membered saturated carbocyclic or heterocyclic ring having from 0 to 2 N atoms

which

a) may contain a double bond and to which

b) may be fused a benzene ring or a saturated or aromatic heterocyclic ring having 1-2 N atoms,

X is CONR³, CH₂CONR³, CH₂NR³, CONR³CH₂, CH₂O, CH₂OCH₂ or OCH₂;

in Iq R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH or an sp²-hybridised carbon atom,

E together with W is a 3- to 7-membered saturated carbocyclic or heterocyclic ring having from 0 to 2 N atoms

which

a) may contain a double bond

and to which

b) may be fused a benzene ring or a saturated or aromatic heterocyclic ring having 1-2 N atoms,

X is CONR³, CH₂CONR³, CH₂NR³, CONR³CH₂, CH₂O, CH₂OCH₂ or OCH₂,

Y is Ar-diyl, and

Ar is phenyl;

in Ir R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH or an sp²-hybridised carbon atom,

E together with W is a 3- to 7-membered saturated carbocyclic or heterocyclic ring having from 0 to 2 N atoms

which

a) may contain a double bond

and to which

b) may be fused a benzene ring or a saturated or aromatic heterocyclic ring having 1-2 N atoms,

X is CONR³, CH₂CONR³, CH₂NR³, CONR³CH₂, CH₂O, CH₂OCH₂ or OCH₂,

Y is Ar-diyl,

Ar is phenyl which is unsubstituted or monosubstituted or disubstituted by Hal, OR⁴, SO₂NH₂, SO₂A or NHCONH₂,

T is a monocyclic or bicyclic, saturated or unsaturated heterocyclic ring having 1 or 2 N and/or O atoms, which is monosubstituted or disubstituted by carbonyl oxygen;

in Is R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH or an sp²-hybridised carbon atom,

E together with W is a 3- to 7-membered saturated carbocyclic or heterocyclic ring having from 0 to 2 N atoms

which

a) may contain a double bond

and to which

b) may be fused a benzene ring or a saturated or aromatic heterocyclic ring having 1-2 N atoms,

X is CONR³, CH₂CONR³, CH₂NR³, CONR³CH₂, CH₂O, CH₂OCH₂ or OCH₂,

Y is Ar-diyl,

Ar is phenyl, which is unsubstituted or monosubstituted or disubstituted by Hal, OR⁴, SO₂NH₂, SO₂A or NHCONH₂,

T is 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2,6-dioxopiperidin-1-yl, 2-oxopiperazin-1-yl, 2,5-dioxopyrrolidin-1-yl, 2-oxo-1,3-oxazolidin-3-yl or 3-oxo-2H-pyridazin-2-yl;

in It R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH or an sp²-hybridised carbon atom,

E together with W is a 3- to 7-membered saturated carbocyclic or heterocyclic ring having from 0 to 2 N atoms

which

a) may contain a double bond

and to which

b) may be fused a benzene ring or a saturated or aromatic heterocyclic ring having 1-2 N atoms,

X is CONR³, CH₂CONR³, CH₂NR³, CONR³CH₂, CH₂O, CH₂OCH₂ or OCH₂,

Y is 1,4-phenylene,

T is 2-oxopiperidin-1-yl, 2-oxopyrrolidin-1-yl, 2-oxo-1 H-pyridin-1-yl, 3-oxomorpholin-4-yl, 4-oxo-1H-pyridin-1-yl, 2,6-dioxopiperidin-1-yl, 2-oxopiperazin-1-yl, 2,5-dioxopyrrolidin-1-yl, 2-oxo-1,3-oxazolidin-3-yl or 3-oxo-2H-pyridazin-2-yl;

in Iu R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH or an sp²-hybridised carbon atom,

E together with W is a 3- to 7-membered saturated carbocyclic or heterocyclic ring having from 0 to 2 N atoms

which

a) may contain a double bond

and to which

b) may be fused a benzene ring or a saturated or aromatic heterocyclic ring having 1-2 N atoms,

X is CONH, CONHCH₂, CH₂NH or CH₂O,

Y is 1,4-phenylene,

T is 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2,6-dioxopiperidin-1-yl, 2-oxopiperazin-1-yl, 2,5-dioxopyrrolidin-1-yl, 2-oxo-1,3-oxazolidin-3-yl or 3-oxo-2H-pyridazin-2-yl;

in Iv R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH or an sp²-hybridised carbon atom,

E together with W is a 3- to 7-membered saturated carbocyclic or heterocyclic ring having from 0 to 2 N atoms

which

a) may contain a double bond

and to which

b) a benzene ring or a saturated or aromatic heterocyclic ring having 1-2 N atoms may be fused,

X is CONH, CONHCH₂, CH₂NH or CH₂O,

Y is 1,4-phenylene,

T is 2-oxopiperidin-1-yl, 2-oxopyrrolidin-1-yl, 2-oxo-1H-pyridin-1-yl, 4-oxo-1H-pyridin-1-yl, 2-oxopiperazin-1-yl, 2- or 3-oxo-2H-pyridazin-2-yl;

in Iw R¹ is CONH₂, CH₂NH₂, or —C(═NH)—NH₂, which is unsubstituted or monosubstituted by OH,

R² is H,

R^{2′} is H, Hal, A, ═CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH or an sp²-hybridised carbon atom,

E together with W is a 3- to 7-membered saturated carbocyclic or heterocyclic ring having from 0 to 2 N atoms

which

a) may contain a double bond

and to which

b) may be fused a benzene ring or a saturated or aromatic heterocyclic ring having 1-2 N atoms,

X is CONH, CONHCH₂, CH₂NH or CH₂O,

Y is 1,4-phenylene,

T is 2-oxopiperidin-1-yl, 2-oxopyrrolidin-1-yl, 2-oxo-1H-pyridin-1-yl, 4-oxo-1H-pyridin-1-yl, 2-oxopiperazin-1-yl, 2-or 3-oxo-2H-pyridazin-2-yl or 2-azabicyclo[2.2.2]octan-3-on-2-yl,

A is unbranched or branched alkyl having 1-6 carbon atoms, and in addition 1-7H atoms may be replaced by F,

Hal is F, Cl or Br;

in Ix R¹ is CONH₂, CH₂NH₂, or —C(═NH)—NH₂, which is unsubstituted or monosubstituted by OH or COOR³,

R² is H,

R^{2′} is H, Hal, A, ═CH—COOA, ═CH—CONH₂ or O—CH₂—COOH,

R^{2″} is H,

R³ is H, alkyl having 1, 2, 3, 4, 5 or 6 carbon atoms, phenyl or benzyl,

W is N or CH or an sp²-hybridised carbon atom,

E together with W is a 3- to 7-membered saturated carbocyclic or heterocyclic ring having from 0 to 2 N atoms

which

a) may contain a double bond

and to which

b) may be fused a benzene ring or a saturated or aromatic heterocyclic ring having 1-2 N atoms,

which

c) may be substituted by carbonyl oxygen,

X is CONH, CONHCH₂, CH₂NH or CH₂O,

Y is 1,4-phenylene,

T is 2-oxopiperidin-1-yl, 2-oxopyrrolidin-1-yl, 2-oxo-1H-pyridin-1-yl, 4-oxo-1H-pyridin-1-yl, 2-oxopiperazin-1-yl, 2-or 3-oxo-2H-pyridazin-2-yl or 2-azabicyclo[2.2.2]octan-3-on-2-yl,

A is unbranched or branched alkyl having 1-6 carbon atoms, and in addition 1-7H atoms may be replaced by F,

Hal is F, Cl or Br;

and pharmaceutically tolerated salts, solvates and stereoisomers thereof.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ so that they are not isolated from the reaction mixture, but instead are immediately converted further into the compounds of the formula 1.

Compounds of the formula I can preferably be obtained by liberating compounds of the formula I from one of their functional derivatives by treatment with a solvolysing or hydrogenolysing agent.

Preferred starting materials for the solvolysis or hydrogenolysis are those which conform to the formula 1, but contain corresponding protected amino and/or hydroxyl groups instead of one or more free amino and/or hydroxyl groups, preferably those which carry an amino-protecting group instead of an H atom bonded to an N atom, in particular those which carry an R′—N group, in which R′ is an amino-protecting group, instead of an HN group, and/or those which carry a hydroxyl-protecting group instead of the H atom of a hydroxyl group, for example those which conform to the formula 1, but carry a —COOR″ group, in which R″ is a hydroxyl-protecting group, instead of a —COOH group.

Preferred starting materials are also the oxadiazole derivatives, which can be converted into the corresponding amidino compounds.

The amidino group can be liberated from its oxadiazole derivative by, for example, treatment with hydrogen in the presence of a catalyst (for example Raney nickel). Suitable solvents are those indicated below, in particular alcohols, such as methanol or ethanol, organic acids, such as acetic acid or propionic acid, or mixtures thereof. The hydrogenolysis is generally carried out at temperatures between about 0 and 1000 and pressures between about 1 and 200 bar, preferably at 20-30° (room temperature) and 1-10 bar.

The oxadiazole group is introduced, for example, by reaction of the cyano compounds with hydroxylamine and reaction with phosgene, dialkyl carbonate, chloroformic acid esters, N,N′-carbonyldiimidazole or acetic anhydride.

It is also possible for a plurality of—identical or different—protected amino and/or hydroxyl groups to be present in the molecule of the starting material.

If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; aralkoxycarbonyl, such as CBZ (“carbobenzoxy”), 4-methoxybenzyloxycarbonyl and FMOC; and arylsulfonyl, such as Mtr. Preferred amino-protecting groups are BOC and Mtr, furthermore CBZ, Fmoc, benzyl and acetyl.

The term “hydroxyl-protecting group” is likewise known in general terms and relates to groups which are suitable for protecting a hydroxyl group against chemical reactions, but are easily removable after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50°, preferably between 15 and 30° (room temperature).

The BOC, OBut and Mtr groups can, for example, preferably be cleaved off using TFA in dichloromethane or using approximately 3 to 5N HCl in dioxane at 15-30°, and the FMOC group can be cleaved off using an approximately 5 to 50% solution of dimethylamine, diethylamine or piperidine in DMF at 15-30°.

Protecting groups which can be removed hydrogenolytically (for example CBZ, benzyl or the liberation of the amidino group from its oxadiazole derivative) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° and pressures between about 1 and 200 bar, preferably at 20-30° and 1-10 bar. Hydrogenolysis of the CBZ group succeeds well, for example, on 5 to 10% Pd/C in methanol or using ammonium formate (instead of hydrogen) on Pd/C in methanol/DMF at 20-30°.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NM P) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

A cyano group is converted into an amidino group by reaction with, for example, hydroxylamine followed by reduction of the N-hydroxyamidine using hydrogen in the presence of a catalyst, such as, for example, Pd/C. In order to prepare an amidine of the formula 1, it is also possible to adduct ammonia onto a nitrile. The adduction is preferably carried out in a number of steps by, in a manner known per se, a) converting the nitrile into a thioamide using H₂S, converting the thioamide into the corresponding S-alkylimidothioester using an alkylating agent, for example CH₃₁, and reacting the thioester in turn with NH₃ to give the amidine, b) converting the nitrile into the corresponding imidoester using an alcohol, for example ethanol in the presence of HCl, and treating the imidoester with ammonia (Pinner synthesis), or c) reacting the nitrile with lithium bis(trimethylsilyl)amide, and subsequently hydrolysing the product.

Esters can be saponified, for example, using acetic acid or using NaOH or KOH in water, water/THF or water/dioxane, at temperatures between 0 and 100°.

Free amino groups can furthermore be acylated in a conventional manner using an acid chloride or anhydride or alkylated using an unsubstituted or substituted alkyl halide, or reacted with CH₃—C(═NH)—Oet, advantageously in an inert solvent, such as dichloromethane or THF and/or in the presence of a base, such as triethylamine or pyridine, at temperatures between −60 and +30″.

If desired, the starting materials can also be formed in situ so that they are not isolated from the reaction mixture, but instead are immediately converted further into the compounds of the formula 1.

Compounds of the formula I in which free NH and/or OH groups are in protected form can preferably be obtained by reacting compounds of the formula II with compounds of the formula III or by reactying compounds of the formula IV with compounds of the formula V. The reaction is generally carried out in an inert solvent, in the presence of an acid-binding agent, preferably an alkali or alkaline earth metal hydroxide, carbonate or bicarbonate, or in the presence of another salt of a weak acid of the alkali or alkaline earth metals, preferably of potassium, sodium, calcium or caesium. The addition of an organic base, such as triethylamine, dimethylaniline, pyridine or quinoline, may also be favourable. Depending on the conditions used, the reaction time is between a few minutes and 14 days, and the reaction temperature is between about 0° and 150°, normally between 20° and 130°.

Examples of suitable inert solvents are water; hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

The starting compounds of the formulae II, III, IV and V are generally known. If they are novel, however, they can be prepared by methods known per se.

In the compounds of the formula II and V, L is preferably Cl, Br, I or a reactively modified OH group, such as, for example, an activated ester, an imidazolide or alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy).

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

On the other hand, compounds of the formula I can be converted into the corresponding metal salts, in particular alkali metal or alkaline earth metal salts, or into the corresponding ammonium salts using bases (for example sodium hydroxide, potassium hydroxide, sodium carbonate or potassium carbonate).

It is also possible to use physiologically acceptable organic bases, such as, for example, ethanolamine.

Compounds of the formula I according to the invention may be chiral owing to their molecular structure and may accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitable N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

The invention furthermore relates to the use of the compounds of the formula I and/or their physiologically acceptable salts for the preparation of pharmaceutical preparations, in particular by non-chemical methods. They can be converted here into a suitable dosage form together with at least one solid, liquid and/or semi-liquid excipient or assistant and, if desired, in combination with one or more further active ingredients.

The invention furthermore relates to medicaments comprising at least one compound of the formula I and/or its pharmaceutically usable derivatives, solvates and stereoisomers, including mixtures thereof in all ratios, and, if desired, excipients and/or assistants.

The invention furthermore relates to pharmaceutical preparations comprising at least one compound of the formula I and/or one of its physiologically acceptable salts.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatin, carbohydrates, such as lactose or starch, magnesium stearate, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders or also as nasal sprays. The novel compounds may also be lyophilised and the resultant lyophilisates used, for example, to prepare injection preparations. The preparations indicated may be sterilised and/or comprise assistants, such as lubricants, preservatives, stabilisers and/or wetting agents, emulsifying agents, salts for modifying the osmotic pressure, buffer substances, colorants and flavours and/or a plurality of further active ingredients, for example one or more vitamins.

The compounds of the formula I and their physiologically acceptable salts can be used for combating and preventing thromboembolic disorders, such as thrombosis, myocardial infarction, arteriosclerosis, inflammation, apoplexia, angina pectoris, restenosis after angioplasty, claudicatio intermittens, tumours, tumour diseases and/or tumour metastases.

In general, the substances according to the invention are preferably administered in doses between about 1 and 500 mg, in particular between 5 and 100 mg, per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

The invention furthermore relates to medicaments comprising at least one compound of the formula I and/or its pharmaceutically usable derivatives, solvates and stereoisomers, including mixtures thereof in all ratios, and at least one further medicament active ingredient.

The invention also relates to a set (kit) consisting of separate packs of

(a) an effective amount of a compound of the formula I and/or its pharmaceutically usable derivatives, solvates and stereoisomers, including mixtures thereof in all ratios, and

(b) an effective amount of a further medicament active ingredient.

The set comprises suitable containers, such as boxes, individual bottles, bags or ampoules. The set may, for example, comprise separate ampoules each containing an effective amount of a compound of the formula I and/or its pharmaceutically usable derivatives, solvates and stereoisomers, including mixtures thereof in all ratios,

and an effective amount of a further medicament active ingredient in dissolved or lyophilised form.

The invention furthermore relates to the use of compounds of the formula I and/or their pharmaceutically usable derivatives, solvates and stereoisomers, including mixtures thereof in all ratios,

for the preparation of a medicament for the treatment of thrombosis, myocardial infarction, arteriosclerosis, inflammation, apoplexia, angina pectoris, restenosis after angioplasty, claudicatio intermittens, tumours, tumour diseases and/or tumour metastases,

in combination with at least one further medicament active ingredient.

Above and below, all temperatures are given in ° C. In the following examples, “conventional work-up” means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent: ethyl acetate/methanol 9:1.

Mass spectrometry (MS): EI (electron impact ionisation) M⁺

FAB (fast atom bombardment) (M+H)⁺

ESI (electrospray ionisation) (M+H)⁺(unless specified otherwise)

### EXAMPLE 1

Preparation of Starting Materials

1.1 Preparation of 1-(4-aminophenyl)piperidin-2-one

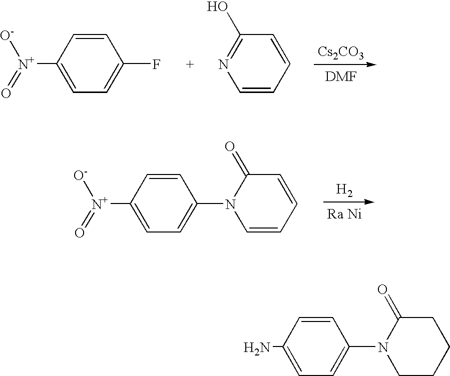

11.5 g (35.3 mmol) of caesium carbonate are added to a solution of 5.00 g (35.4 mmol) of 1-fluoro-4-nitrobenzene and 3.40 g (35.7 mmol) of 2-pyridinol in 50 ml of DMF, and the mixture is heated at 110° C. for 24 hours. The reaction mixture is allowed to cool, and is poured into water. The precipitate is filtered off, dried and recrystallised from ethyl acetate, giving 1-(4-nitrophenyl)-1H-pyridin-2-one as a yellowish solid; ESI 217.

1.5 g of water-moist Raney nickel are added to a solution of 4.60 g (21.3 mmol) of 1-(4-nitrophenyl)-1H-pyridin-2-one in 150 ml of methanol, and the mixture is hydrogenated at room temperature and atmospheric pressure for 22 hours. The reaction mixture is filtered, and the filtrate is evaporated, giving 1-(4-aminophenyl)piperidin-2-one as a colourless solid; ESI 191.

1.2 Preparation of 4-(tert-butoxycarbonyl)-1-(3-cyanophenyl)piperazine-2-carboxylic Acid

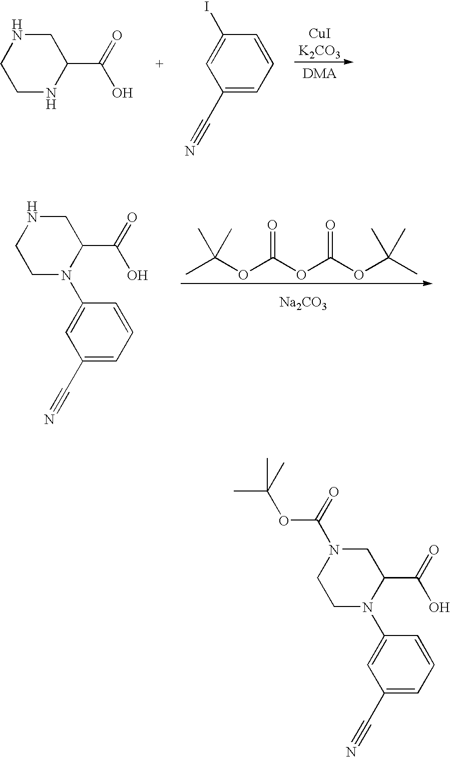

346 mg (2.50 mmol) of potassium carbonate and 19 mg (0.10 mmol) of copper(I) iodide are added to a solution of 203 mg (1.00 mmol) of piperazine-2-carboxylic acid dihydrochloride and 229 mg (1.00 mmol) of 3-iodobenzonitrile in 2 ml of N,N-dimethylacetamide, and the mixture is heated at 200° C. for 5 minutes in a closed vessel in a microwave unit. After the reaction mixture has cooled, ether is added, and the resultant precipitate is filtered off, giving crude 1-(3-cyanophenyl)piperazine-2-carboxylic acid as the potassium salt; ESI 232.

The crude product obtained in this way, 218 mg (1.00 mmol) of di-tert-butyl dicarbonate and 106 mg (1.00 mmol) of sodium carbonate are dissolved in 10 ml of dioxane and 5 ml of water, and the mixture is stirred at room temperature for 18 hours. The reaction mixture is evaporated and partitioned between water and diethyl ether. The aqueous phase is acidified using 1 N HCl and extracted with diethyl ether. The organic phase is dried over sodium sulfate and evaporated, giving 4-(tert-butoxycarbonyl)-1-(3-cyanophenyl)piperazine-2-carboxylic acid as a colourless solid; ESI 353 (M+Na⁺).

1.3 Preparation of 1-(3-cyanophenyl)piperidine-2-carboxylic Acid

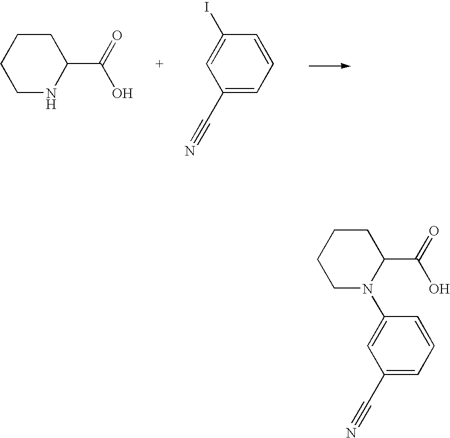

1.5 g (1.3 mmol) of tetrakis(triphenylphosphine)palladium(0), 0.25 g (1.3 mmol) of copper(I) iodide, 3.6 g (26 mmol) of potassium carbonate and 1.6 g (4.4 mmol) of tetrabutylammonium iodide are added to a solution of 3.36 g (26.0 mmol) of piperidine-2-carboxylic acid and 5.96 g (26.0 mmol) of 3-iodobenzonitrile in 20 ml of pyridine, 50 ml of 1-methyl2-pyrrolidone and 5 ml of water, and the mixture is stirred at 1001° C. for 19 hours. The reaction mixture is partitioned between 1 N HCl and ethyl acetate, and the organic phase is extracted with 10% sodium carbonate solution. The aqueous phase is adjusted to a pH of 2.5 using 25% HCl and extracted with ethyl acetate. The organic phase is dried over sodium sulfate and evaporated, giving 1-(3-cyanophenyl)piperidine-2-carboxylic acid as a colourless oil; ESI 231.

1.4 Preparation of 2-(3-cyanophenyl)cyclopent-1-enecarboxylic Acid

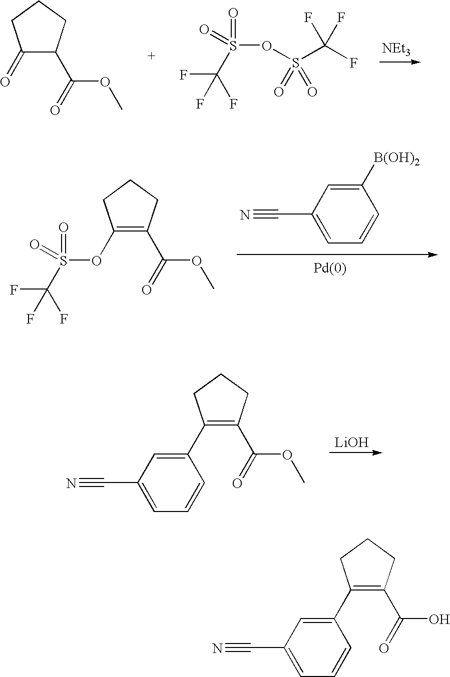

21.1 ml (152 mmol) of triethylamine are slowly added at 0° C. to a solution of 21.3 g (150 mmol) of methyl 2-oxocyclopentanecarboxylate in 400 ml of dichloromethane. A solution of 25 ml (152 mmol) of trifluoromethanesulfonic anhydride in 100 ml of dichloromethane is added dropwise over the course of one hour at an internal temperature of from −6 to 0° C. The reaction mixture is warmed to room temperature and introduced into water. Extractive work-up gives methyl 2-trifluoromethanesulfonyloxycyclopent-1-enecarboxylate as a colourless oil.

15.9 g (115 mmol) of potassium carbonate and 2.0 g (1.7 mmol) of tetrakis(triphenylphosphine)palladium are added to a solution of 30.0 g (109 mmol) of methyl 2-trifluoromethanesulfonyloxycyclopent-1-enecarboxylate and 16.2 g (110 mmol) of 3-cyanobenzeneboronic acid in a mixture of 300 ml of toluene and 100 ml of methanol, and the mixture is heated at 110° C. for 4 hours. The reaction mixture is cooled to room temperature and introduced into water, and the organic phase is separated off. The organic phase is evaporated and recrystallised from petroleum ether, giving methyl 2-(3-cyanophenyl)cyclopent-1-enecarboxylate as a colourless solid; ESI 228.

A solution of 5.00 g (22.0 mmol) of methyl 2-(3-cyanophenyl)cyclopent-1-enecarboxylate and 790 mg (33.0 mmol) of lithium hydroxide in a mixture of 50 ml of methanol and 50 ml of water is stirred at room temperature for 18 hours. The reaction mixture is evaporated, and the residue is extracted with ethyl acetate. The aqueous phase is acidified, and the resultant precipitate is filtered off, giving 2-(3-cyanophenyl)cyclopent-1-enecarboxylic acid as a colourless solid; ESI 214.

The following carboxylic acid building blocks were prepared analogously:

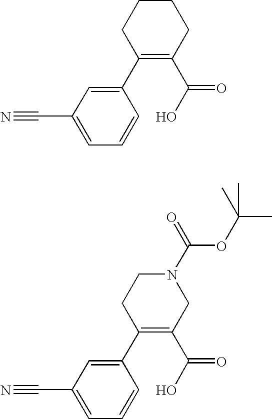

1.5 Preparation of trans-2-(3-cyanophenyl)cyclopentanecarboxylic Acid

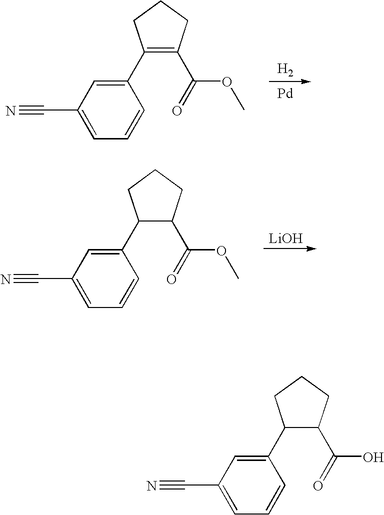

500 mg of palladium on activated carbon are added to a solution of 4.00 g (17.6 mmol) of methyl 2-(3-cyanophenyl)cyclopent-1-enecarboxylate in 50 ml of methanol, and the mixture is hydrogenated. The catalyst is filtered off, and the filtrate is evaporated, giving methyl 2-(3-cyanophenyl)cyclopentanecarboxylate; ESI 230.

A solution of 2.80 g (12.2 mmol) of methyl 2-(3-cyanophenyl)cyclopentanecarboxylate and 455 mg (19.0 mmol) of lithium hydroxide in a mixture of 30 ml of methanol and 30 ml of water is stirred at room temperature for 18 hours. The reaction mixture is evaporated, and the residue is extracted with ethyl acetate. The aqueous phase is acidified, and the resultant precipitate is filtered off, giving trans-2-(3-cyanophenyl)cyclopentanecarboxylic acid as a colourless solid; ESI 216.

### EXAMPLE 2

Preparation of N-[4-(2-oxopiperidin-1-yl)phenyl]-1-(3-carbamoylphenyl)piperazine-2-carboxamide

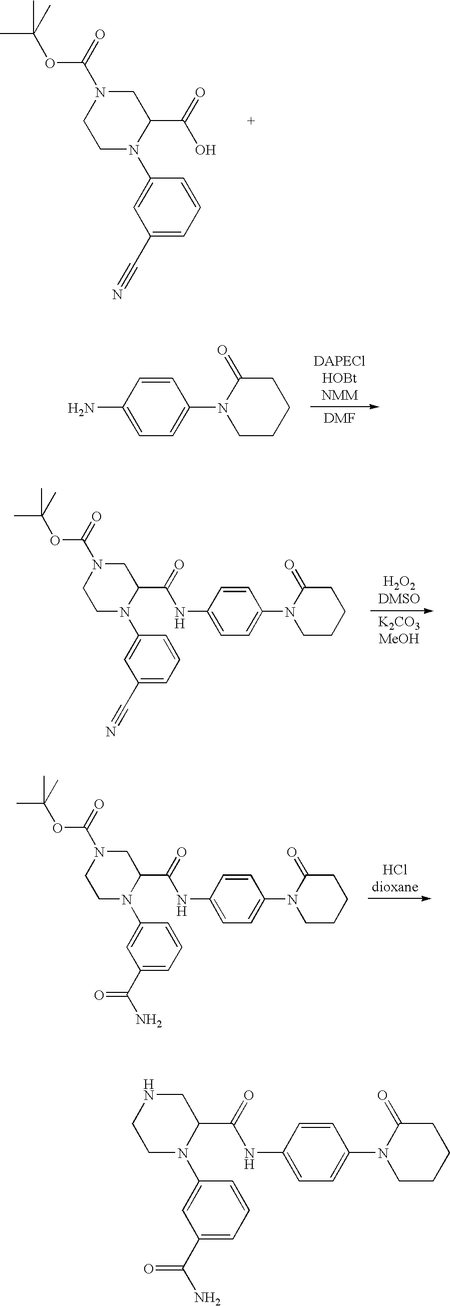

33 μl (0.30 mmol) of 4-methylmorpholine are added to a solution of 100 mg (0.302 mmol) of 4-(tert-butoxycarbonyl)-1-(3-cyanophenyl)piperazine-2-carboxylic acid, 57.5 mg (0.302 mmol) of 1-(4-amino-phenyl)piperidin-2-one, 57.9 mg (0.302 mmol) of N-(3-dimethylaminopropyl)-N′-ethylcarbodiimide hydrochloride (DAPECI) and 40.8 mg (0.302 mmol) of hydroxybenzotriazole hydrate (HOBt) in 1 ml of DMF, and the mixture is stirred at room temperature for 18 hours. The reaction mixture is introduced into water, and the precipitate is filtered off, giving tert-butyl 4-(3-cyanophenyl)-3-[4-(2-oxopiperidin-1-yl)phenylcarbamoyl]piperazine-1-carboxylate as a colourless solid; ESI 447 (M−tBu)⁺.

61 μl (0.87 mmol) of dimethyl sulfoxide, 170 mg (1.24 mmol) of potassium carbonate and 0.126 ml (1.24 mmol) of 30% hydrogen peroxide are added to a solution of 100 mg (0.199 mmol) of tert-butyl 4-(3-cyanophenyl)-3-[4-(2-oxopiperidin-1-yl)phenylcarbamoyl]piperazine-1 carboxylate in 1 ml of methanol, and the mixture is stirred at room temperature for 2 hours. The reaction mixture is partitioned between water and ethyl acetate. The organic phase is evaporated, and the residue is chromatographed on a silica-gel column with petroleum ether/ethyl acetate as eluent, giving tert-butyl 4-(3-carbamoylphenyl)-3-[4-(2-oxopiperidin-1-yl)phenylcarbamoyl]piperazine-1-carboxylate as a colourless solid; ESI 522.

44 mg (0.084 mmol) of tert-butyl 4-(3-carbamoylphenyl)-3-[4-(2-oxopiperidin-1-yl)phenylcarbamoyl]piperazine-1-carboxylate are dissolved in 2.0 g of 4N HCl in dioxane, and the mixture is left to stand for 1 hour and evaporated, giving N-[4-(2-oxopiperidin-1-yl)phenyl]-1-(3-carbamoylphenyl)piperazine-2-carboxamide hydrochloride as a colourless solid; ESI 422.

The following compounds are obtained analogously:

N-[4-(2-oxopiperidin-1-yl)phenyl]-1-(3-carbamoylphenyl)piperidine-2-carboxamide, ESI 421;

N-[4-(2-oxopiperidin-1-yl)phenyl]-1-(3-carbamoylphenyl)pyrrolidine-2-carboxamide,

N-[4-(2-oxopiperidin-1-yl)phenyl]-1-(3-carbamoylphenyl)-2,3-dihydro-1H-isoindole-1-carboxamide,

N-[4-(2-oxopiperazin-1-yl)phenyl]-1-(3-carbamoylphenyl)piperidine-2-carboxamide, N-[4-(2-oxopyridin-1-yl)phenyl]-1-(3-carbamoylphenyl)piperidine-2-carboxamide,

N-[4-(2-oxopiperidin-1-yl)phenyl]-1-(3-carbamoylphenyl)-4-(2,2,2-trifluoroethyl)piperidinecarboxamide,

N-[4-(2-oxopiperidin-1-yl)phenylmethyl]-1-(3-carbamoylphenyl)piperidine-2-carboxamide.

### EXAMPLE 3

Cyclopentene and Cyclopentane Derivatives are Obtained in Accordance with the Following Reaction Scheme:

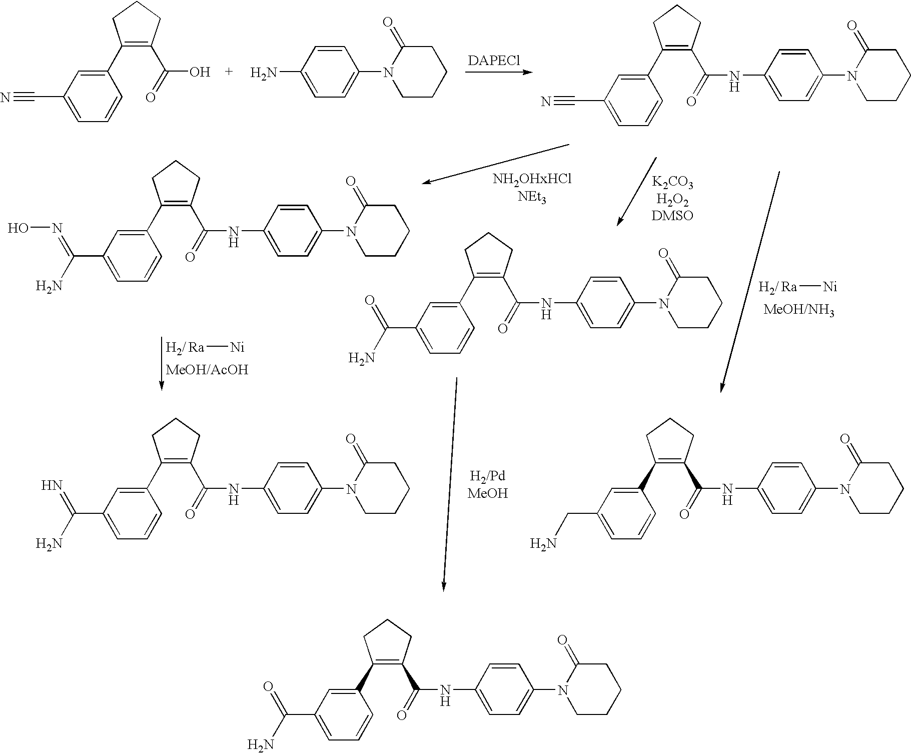

0.11 ml (1.0 mmol) of 4-methylmorpholine is added to a solution of 215 mg (1.00 mmol) of 2-(3-cyanophenyl)cyclopent-1-enecarboxylic acid, 190 mg (1.00 mmol) of 1-(4-aminophenyl)piperidin-2-one, 192 mg (1.00 mmol) of N-(3-dimethylaminopropyl)-N′-ethylcarbodiimide hydrochloride (DAPECI) and 135 mg (1.00 mmol) of hydroxybenzotriazole hydrate (HOBt) in 2 ml of DMF, and the mixture is stirred at room temperature for 18 hours. The reaction mixture is introduced into water, and the precipitate is filtered off, giving N-[4-(2-oxopiperidin-1-yl)phenyl]-2-(3-cyanophenyl)cyclopent-1-enecarboxamide as a colourless solid; ESI 388.

0.14 ml (1.0 mmol) of triethylamine is added to a solution of 140 mg (0.363 mmol) of N-[4-(2-oxopiperidin-1-yl)phenyl]-2-(3-cyanophenyl)cyclopent-1-enecarboxamide and 69.5 mg (1.00 mmol) of hydroxyl-ammonium chloride in 8 ml of methanol, and the mixture is heated at 70° C. for 18 hours. The reaction mixture is evaporated and introduced into water. The resultant precipitate is filtered off and chromatographed on a silica-gel column with ethyl acetate/methanol as eluent, giving N-[4-(2-oxopiperidin-1-yl)phenyl]-2-[3-(N-hydroxyamidino)phenyl]cyclopent-1-enecarboxamide as a colourless solid; ESI 419.

30 mg of acetic acid and 100 mg of Raney nickel are added to a solution of 20 mg (0.048 mmol) of N-[4-(2-oxopiperidin-1-yl)phenyl]-2-[3-(N-hydroxycarbamimidoyl)phenyl]cyclopent-1-enecarboxamide in 10 ml of methanol, and the mixture is hydrogenated at room temperature and atmospheric pressure. The catalyst is filtered off, and the filtrate is evaporated, giving N-[4-(2-oxopiperidin-1-yl)phenyl]-2-(3-amidinophenyl)cyclopent-1-enecarboxamide acetate as a colourless solid; ESI 403.

0.24 ml (3.4 mmol) of dimethyl sulfoxide, 680 mg (4.92 mmol) of potassium carbonate and 0.50 ml (4.9 mmol) of 30% hydrogen peroxide are added to a solution of 300 mg (0.778 mmol) of N-[4-(2-oxopiperidin-1-yl)phenyl]-2-(3-cyanophenyl)cyclopent-1-enecarboxamide in 10 ml of methanol. The reaction mixture is stirred at room temperature for 2 hours, subsequently introduced into water and extracted with ethyl acetate. The organic phase is evaporated, giving 3-{2-[4-(2-oxopiperidin-1-yl)phenylcarbamoyl]cyclopent-1-enyl}benzamide as a colourless solid, ESI 404.

100 mg of palladium on activated carbon are added to a solution of 150 mg (0.372 mmol) of 3-{2-[4-(2-oxopiperidin-1-yl)phenylcarbamoyl]-cyclopent-1-enyl}benzamide in 10 ml of methanol, and the mixture is hydrogenated under a slight superatmospheric pressure. The catalyst is filtered off, and the filtrate is evaporated, giving 3-{cis-2-[4-(2-oxo piperidin-1-yl)phenylcarbamoyl]cyclopentyl}benzamide as a colourless solid; ESI 406.

500 mg of Raney nickel are added to a solution of 80.0 mg (0.208 mmol) of N-[4-(2-oxopiperidin-1-yl)phenyl]-2-(3-cyanophenyl)cyclopent-1-enecarboxamide in 40 ml of saturated methanolic ammonia solution, and the mixture is hydrogenated at room temperature and under a slight superatmospheric pressure. The catalyst is filtered off, and the filtrate is evaporated, giving N-[4-(2-oxopiperidin-1-yl)phenyl]-cis-2-(3-aminomethylphenyl)cyclopentanecarboxamide as a yellowish oil. 3 ml of 1 N HCl in isopropanol are added to the crude product obtained in this way, and the mixture is evaporated. The residue is taken up in diethyl ether, and the precipitate is filtered off, giving N-[4-(2-oxopiperidin-1-yl)phenyl]-cis-2-(3-aminomethylphenyl)cyclopentanecarboxamide hydrochloride as a colourless solid; ESI 392.

The following compounds are obtained analogously:

N-[4-(2-oxopiperidin-1-yl)phenyl]-cis-2-(3-aminomethylphenyl)cyclopropanecarboxamide,

N-[4-(2-oxopiperidin-1-yl)phenyl]-2-(3-amidinophenyl)piperidine-2-carboxamide, ESI 420;

N-[4-(2-oxopiperidin-1-yl)phenyl]-2-(3-aminomethylphenyl)piperidine-2-carboxamide, ESI 407;

N-[3-(2-oxopiperidin-1-yl)phenyl]-2-(3-aminomethylphenyl)piperidine-230 carboxamide, ESI 407;

3-{2-[4-(2-oxopiperidin-1-yl)phenylcarbamoyl]cyclohex-1-enyl}benzamide,

3-{2-[4-(2-oxopiperidin-1-yl)phenylcarbamoyl]cyclohexyl}benzamide,

N-[4-(2-oxopiperidin-1-yl)phenyl]-4-(3-carbamoylphenyl)-1,2,5,6-tetrahydropyridine-3-carboxamide,

N-[4-(2-oxopiperidin-1-yl)-2-fluorophenyl]-2-(3-aminomethylphenyl)piperidine-2-carboxamide, ESI 425;

N-[4-(2-oxopiperidin-1-yl)phenyl]-(S)-2-(3-aminomethylphenyl)-5-oxopyrrolidine-2-carboxamide, ESI 407;

N-[4-(2-oxopiperidin-1-yl)phenyl]-(R)-2-(3-aminomethylphenyl)-pyrrolidine-2-carboxamide, ESI 393;

N-[4-(2-oxopiperidin-1-yl)phenyl]-2-[3-(N-hydroxyamidino)phenyl]-piperidine-2-carboxamide, ESI 436;

N-[4-(3-oxo-2-azabicyclo[2.2.2]oct-2-yl)phenyl]-2-[3-(N-hydroxyamidino)phenyl]piperidine-2-carboxamide, ESI 462;

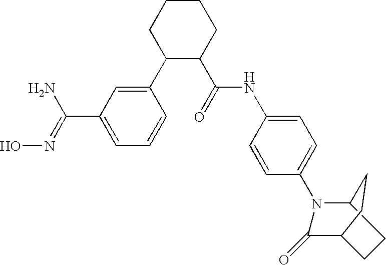

N-[4-(3-oxo-2-azabicyclo[2.2.2]oct-2-yl)phenyl]-2-(3-amidinophenyl)-piperidine-2-carboxamide, ESI 462;

N-[4-(3-oxo-2-azabicyclo[2.2.2]oct-2-yl)phenyl]-2-(3-aminomethylphenyl)-piperidine-2-carboxamide, ESI 433.

### EXAMPLE 4

The compounds

3-{2-[4-(2-oxopiperidin-1-yl)phenylcarbamoyl]cyclopentyl}benzamide and N-[4-(2-oxopiperidin-1-yl)phenyl]-2-(3-aminomethylphenyl)cyclopentane-carboxamide

are obtained in accordance with the following reaction scheme:

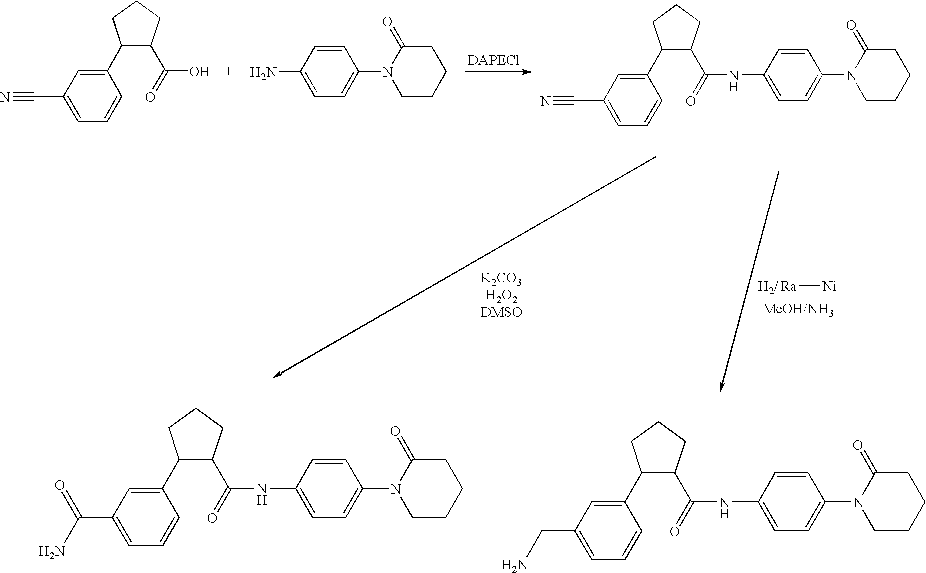

### EXAMPLE 5

The compounds

3-(2-{[4-(2-oxopiperidin-1-yl)phenylamino]methyl}piperidin-1-yl)benzamide and

3-(2-{[4-(2-oxopiperidin-1-yl)phenoxy]methyl}piperidin-1-yl)-benzamide are obtained in accordance with the following reaction schemes:

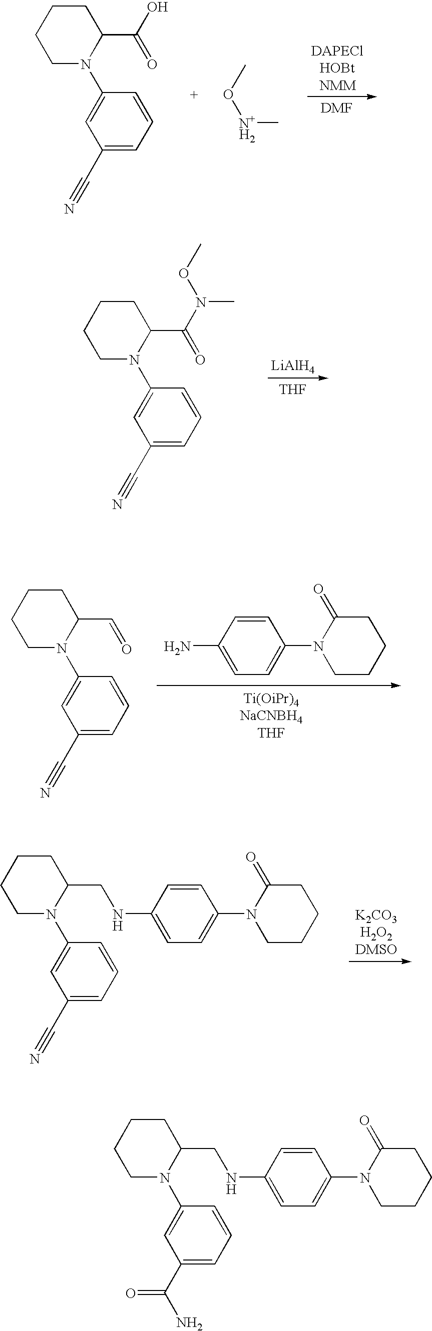

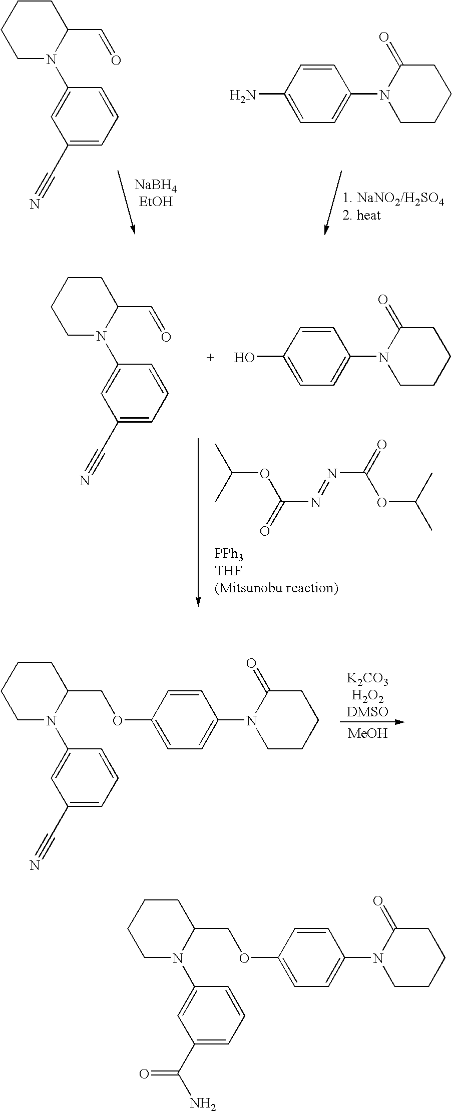

### EXAMPLE 6

The compound {1-(3-carbamoylphenyl)-2-[4-(2-oxopiperidin-1-yl)phenylcarbamoyl]piperidin-4-ylidene}acetic Acid

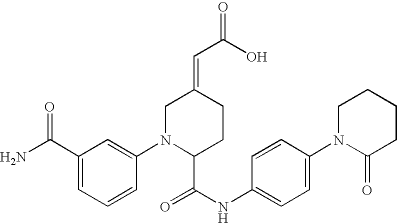

is obtained in accordance with the following reaction scheme:

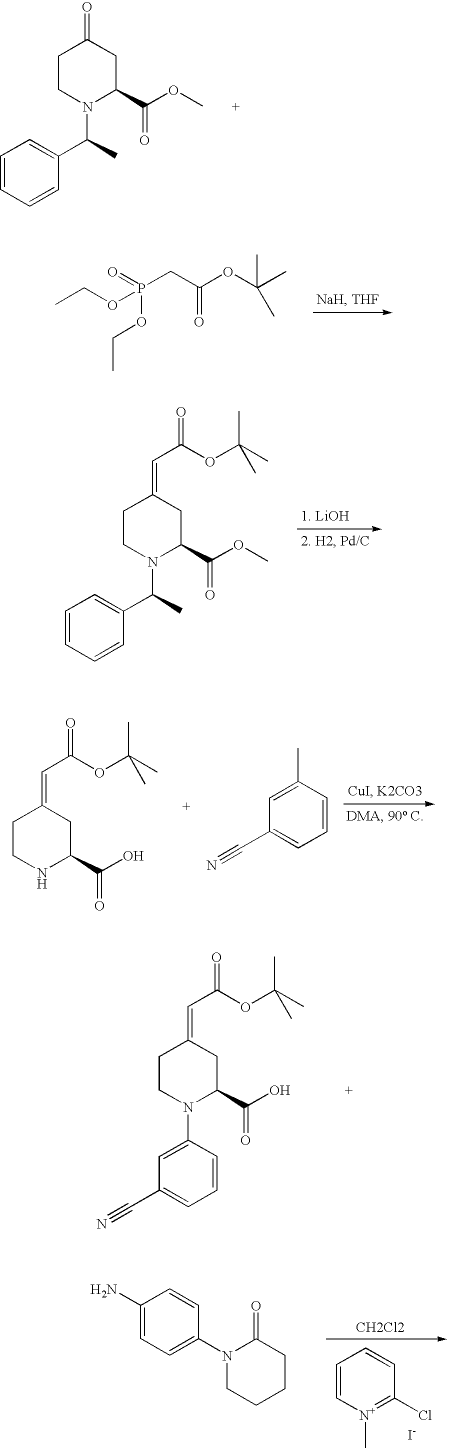

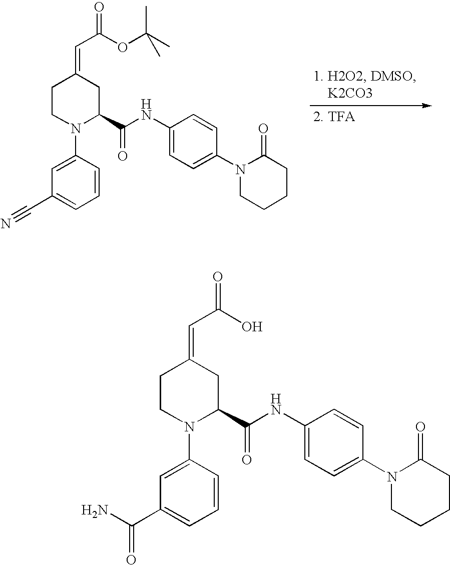

### EXAMPLE 7

An analogous reaction to Example 2 gives {1-(3-carbamoylphenyl)-2-[4-(2-oxopiperidin-1-yl)phenylcarbamoyl]-piperidin-4-yloxy}acetic acid

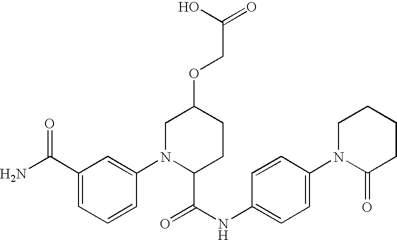

### EXAMPLE 8

The compound N-[4-(2-oxopiperidin-1-yl)phenyl]-5-(3-aminomethylphenyl)-1-methyl-4,5,6,7-tetrahydro-1H-imidazo[4,5-c]pyridine-6-carboxamide

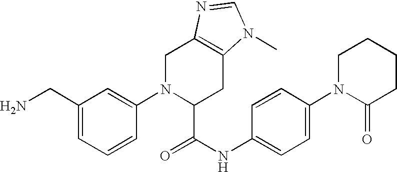

is obtained in accordance with the following reaction scheme:

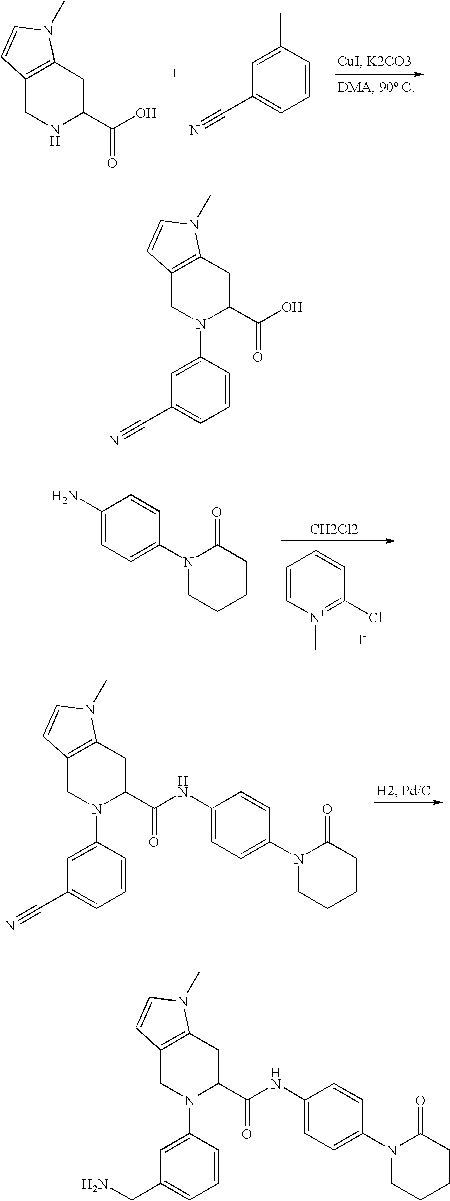

### EXAMPLE 9

The compound {5-(3-carbamoylphenyl)-6-[4-(2-oxopiperidin-1-yl)phenylcarbamoyl]-4,5,6,7-tetrahydroimidazo[4,5-c]pyridin-1-yl}acetic acid

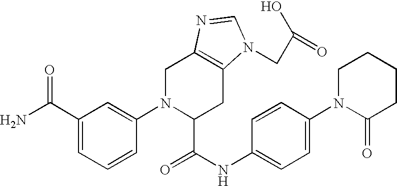

is obtained analogously to Example 2.

### EXAMPLE 10

N-[4-(2-oxopiperidin-1-yl)phenyl]-2-(3-amidinophenyl)piperidine-2-carboxamide gives, by conventional methods, the compound N-[4-(2-oxopiperidin-1-yl)phenyl]-2-[3-(N-ethoxycarbonylamidino)phenyl]-piperidine-2-carboxamide, ESI 492.

11. Examples of the Preparation of Intermediates

11.1 1-(4-aminophenyl)-1H-pyrazin-2-one

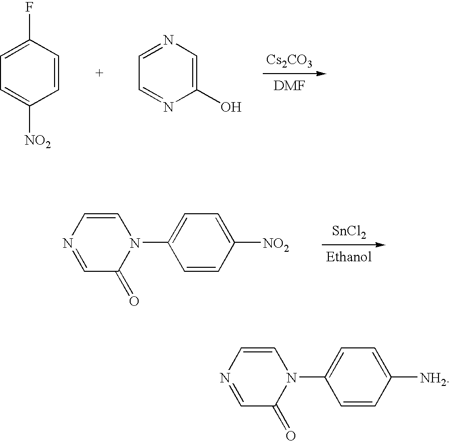

11.2 1-(4-amino-2,5-dimethylphenyl)piperidin-2-one

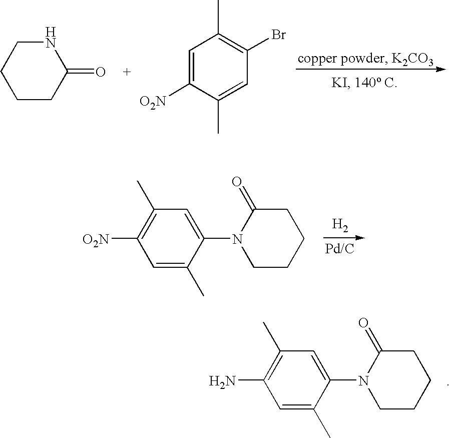

11.3 1-(4-amino-3-methylphenyl)piperidin-2-one

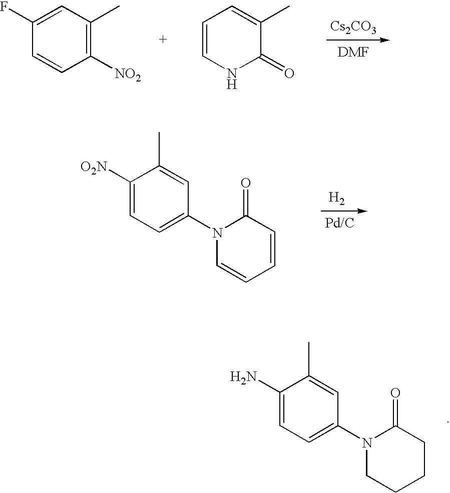

11.4 1-(5-aminopyridin-2-yl)piperidin-2-one

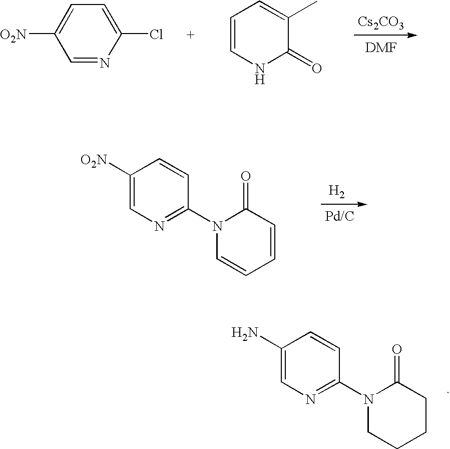

11.5 1-(4-aminomethylphenyl)piperidin-2-one

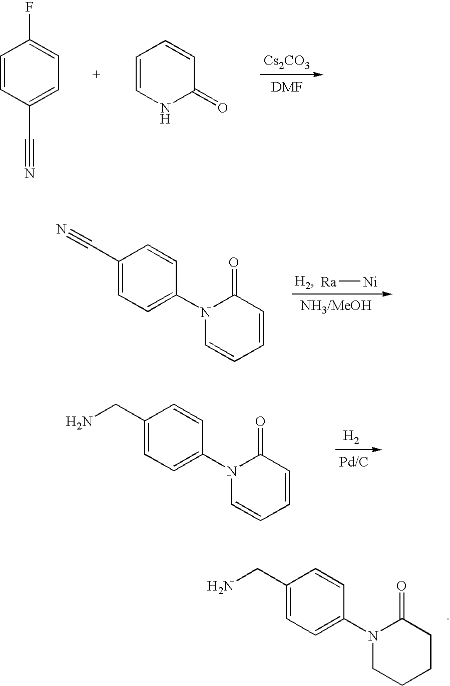

11.6 2-(4-aminophenyl)-2-azabicyclo[2.2.2]octan-3-one

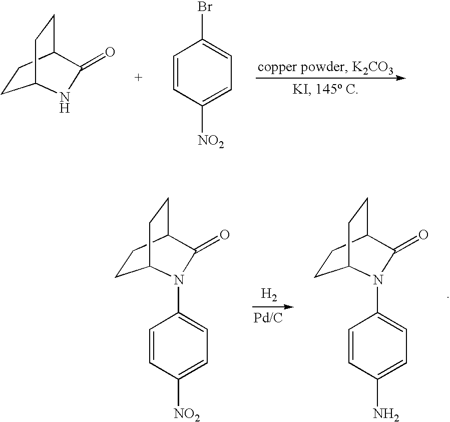

11.7 1-(3-amino-6-ethylphenyl)pyrrolidin-2-one

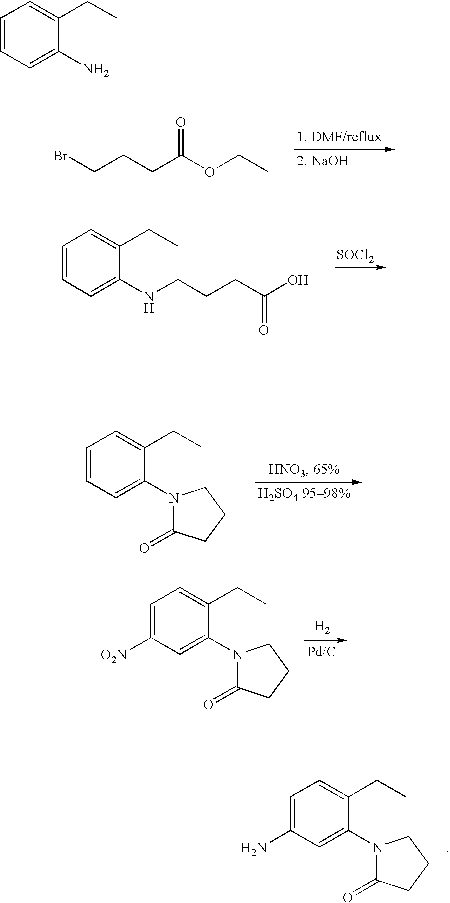

11.8 2-(4-amino-2-trifluoromethylphenyl)-2-azabicyclo[2.2.2]octan-3-one

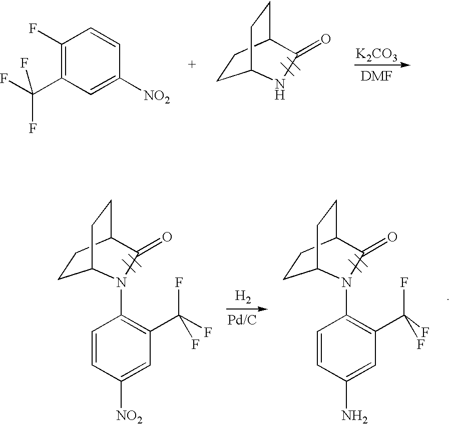

11.9 1-(4-amino-3-chlorophenyl)pyrrolidin-2-one

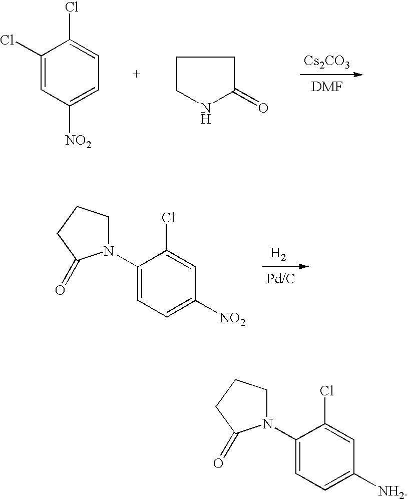

11.11 1-(4-amino-2-trifluoromethylphenyl)piperidin-2-one

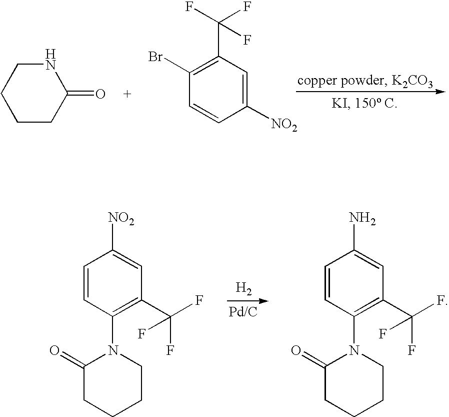

11.12 3-(4-amino-2-methylphenyl)-1,3-oxazinan-2-one

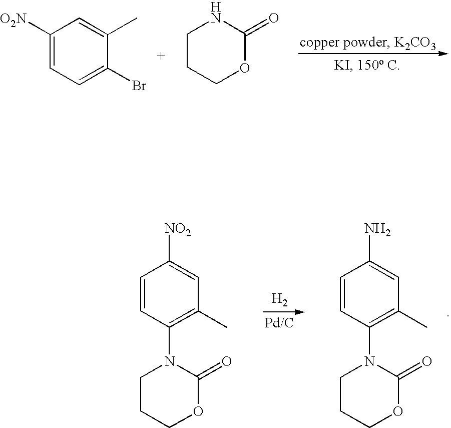

11.13 4-(4-aminophenyl)morpholin-3-one

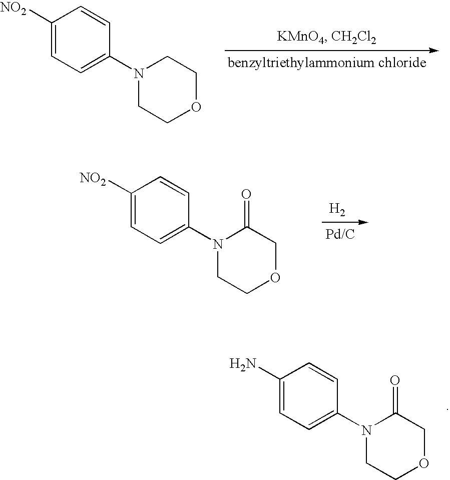

11.14 1-(4-aminophenyl)pyridin-2-one

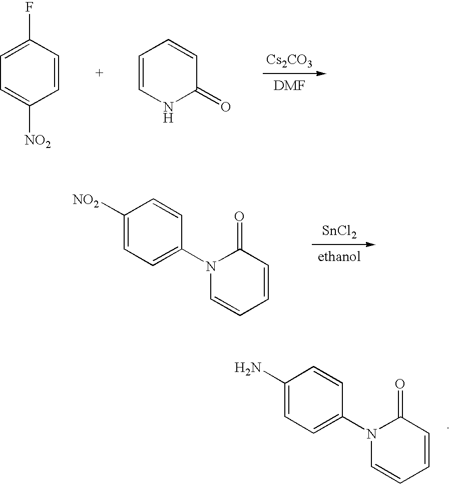

11.15 1-(4-amino-2-methylphenyl)piperidin-2-one

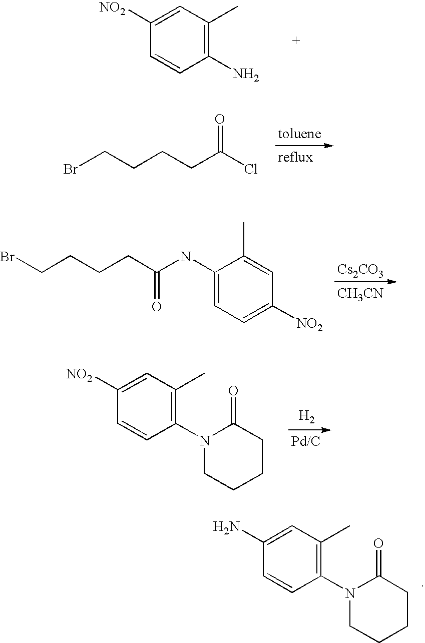

11.16 1-(4-aminophenyl)-1H-pyridin-4-one

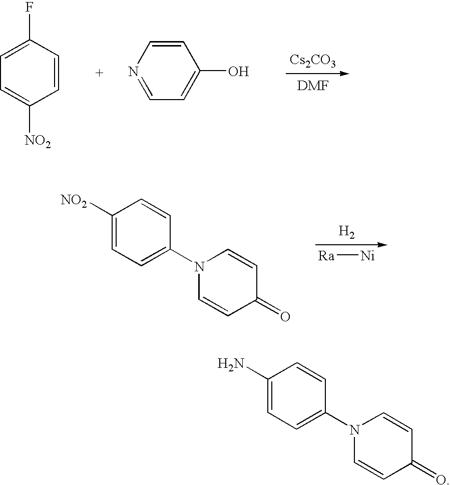

11.17 1-(4-aminophenyl)-4-tert-butoxycarbonylpiperazin-2-one

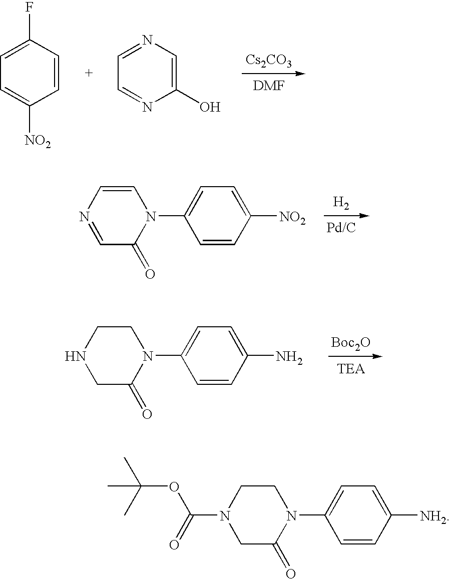

11.18 1-(3-aminophenyl)piperidin-2-one

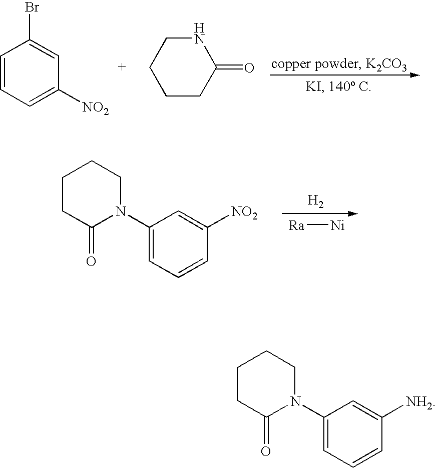

11.19 1-(4-aminophenyl)-2-caprolactam

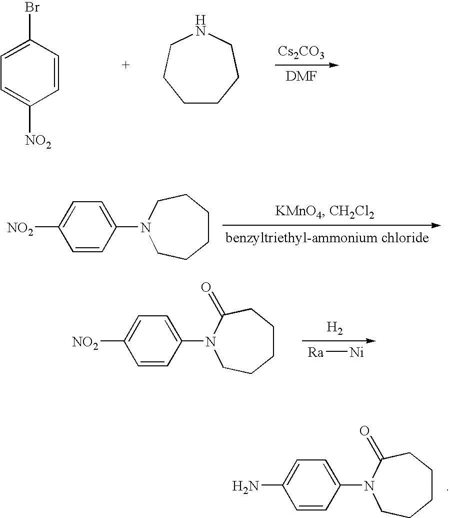

11.20 1-(4-amino-3-fluorophenyl)piperidin-2-one

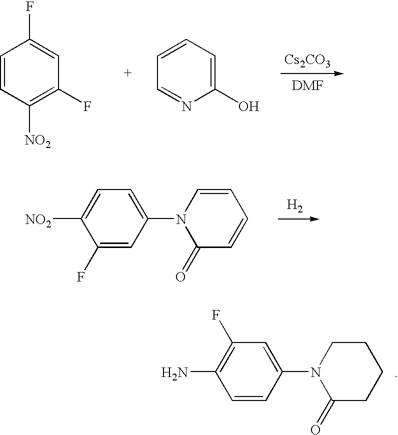

11.21 1-(4-amino-2-fluorophenyl)piperidin-2-one

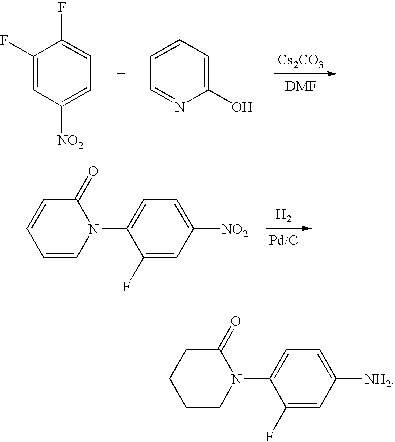

11.22 1-(4-amino-2-fluoro)-2-caprolactam

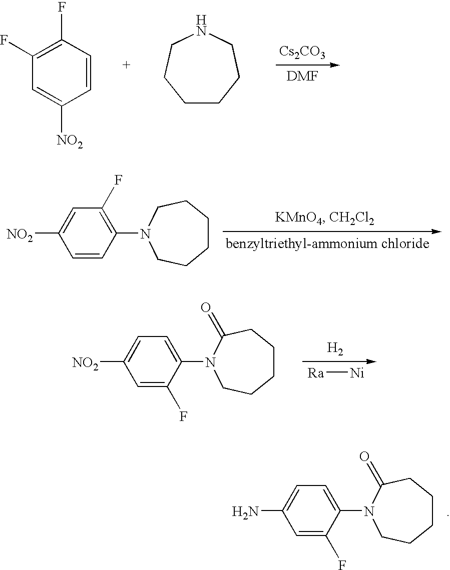

11.23 2-(2-fluorophenyl)-3-(3-cyanophenyl)propionic Acid

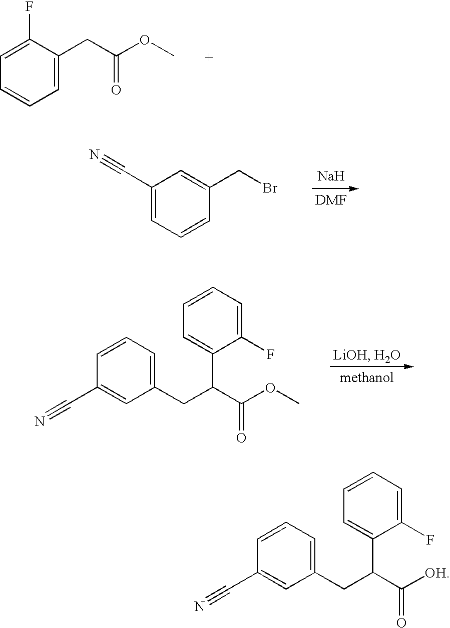

The examples below relate to pharmaceutical preparations:

### EXAMPLE A

### Injection Vials

A solution of 100 g of an active ingredient of the formula 1 and 5 g of disodium hydrogenphosphate in 3 I of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

Solution

A solution is prepared from 1 g of an active ingredient of the formula 1, 9.38 g of NaH₂PO₄·2H₂O, 28.48 g of Na₂HPO₄ 12H₂O and 0.1 g of benzalkonium chloride in 940 ml of bidistilled water. The pH is adjusted to 6.8, and the solution is made up to II and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula 1, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced in a conventional manner into hard gelatine capsules in such a way that each capsule contains 20 mg of the active ingredient.

### EXAMPLE H

### Ampoules

A solution of 1 kg of active ingredient of the formula I in 60 I of bidistilled water is sterile filtered, transferred into ampoules, lyophilised under sterile conditions and sealed under sterile conditions. Each ampoule contains 10 mg of active ingredient.

1. Compounds of the formula I 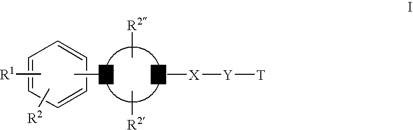
in which
R¹ is H, CN, or is —C(═NH)—NH₂, CON(R³)₂ or —[C(R⁴)₂]ₙN(R³)₂, each of which is unsubstituted or monosubstituted by C(═O)R³, COOR³, OR³ or by a conventional amino-protecting group, or is 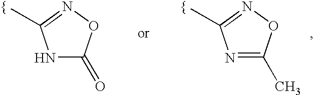
R², R^{2′}
and R^{2″} are each, independently of one another, H, Hal, A, OR³, N(R³)₂, NO₂, CN, —[C(R⁴)₂]ₙ—Ar, —[C(R⁴)₂]ₙ-Het, —[C(R⁴)₂]ₙ-cycloalkyl, ═C(R⁴)—[C(R⁴)₂]ₙ-COOR³═C(R⁴)—[C(R⁴)₂]ₙ—CON(R³)₂, —[C(R⁴)₂]ₙ—COOR³, —[C(R⁴)₂]ₙ—CON(R³)₂, O—[C(R⁴)₂]ₙ, —COOR³
or O—[C(R⁴)₂]ₙ—CON(R³)₂,
R³ is H, A, —[C(R⁴)₂]ₙ—Ar, —[C(R⁴)₂]ₙ-Het or —[C(R⁴)₂]ₙ-cycloalkyl,
R⁴ is H or A,
W is N, CR or an sp²-hybridised carbon atom,
E together with W is a 3- to 7-membered saturated carbocyclic or heterocyclic ring having from 0 to 3 N atoms, from 0 to 2 O atoms and/or from 0 to 2 S atoms, which
a) may contain a double bond
to which
b) may be fused a benzene ring or a saturated, unsaturated or aromatic heterocyclic ring,
which
c) may be substituted by carbonyl oxygen and/or by R² and/or R²,
X is —[C(R⁴)₂]ₙCONR³[C(R⁴)₂]ₙ—, —[C(R⁴)₂]ₙNR³CO[C(R⁴)₂]ₙ—, —[C(R⁴)₂]ₙNR³[C(R⁴)₂]ₙ or —[C(R⁴)₂]ₙO[C(R⁴)₂]ₙ—,
Y is alkylene, cycloalkylene, Het-diyl or Ar-diyl,
T is a monocyclic or bicyclic, saturated or unsaturated heterocyclic ring having from 1 to 4 N, O and/or S atoms, which is monosubstituted or disubstituted by carbonyl oxygen and which may additionally be monosubstituted, disubstituted or trisubstituted by Hal, A, —[C(R⁴)₂]ₙ—Ar, —[C(R⁴)₂]ₙ-Het, —[C(R⁴)₂]ₙ-cycloalkyl, OR³, N(R³)₂, NO₂, CN, COOR³, CON(R³)₂, NR³COA, NR₃CON(R₃)₂, NR³SO₂A, COR³, SO₂NR³ or S(O)ₘA,
A is unbranched or branched alkyl having 1-6 carbon atoms, in which one or two CH₂ groups may be replaced by O or S atoms and/or by —CH═CH— groups and/or, in addition, 1-7H atoms may be replaced by F,
Ar is phenyl, naphthyl or biphenyl, each of which is unsubstituted or monosubstituted, disubstituted or trisubstituted by Hal, A, OR⁴, N(R⁴)₂, NR⁴CON(R⁴)₂, NO₂, CN, COOR⁴, CON(R⁴)₂, NR⁴COA, NR⁴SO₂A, COR⁴, SO₂NR⁴ or S(O)ₘA,
Het is a monocyclic or bicyclic, saturated, unsaturated or aromatic heterocyclic ring having from 1 to 4 N, O and/or S atoms which is unsubstituted or monosubstituted, disubstituted or trisubstituted by Hal, A, —[C(R⁴)₂]ₙ—Ar, —[C(R⁴)₂]ₙ-Het′, [C(R⁴)₂]ₙ-cycloalkyl, —[C(R⁴)₂]ₙ—CON(R³)₂, —[C(R⁴)₂]ₙ—COOR³, OR³, N(R³)₂, NR³CON(R³)₂, NO₂, CN, NR³COA, NR³SO₂A, COR³, SO₂NR³, S(O)ₘA and/or carbonyl oxygen,
Het′ is a monocyclic or bicyclic, saturated, unsaturated or aromatic heterocyclic ring having from 1 to 4 N, O and/or S atoms which is unsubstituted or monosubstituted or disubstituted by Hal, A, OR³, N(R³)₂, NO₂, CN, COOR³, CON(R³)₂, NR³COA, NR³SO₂A, COR³, SO₂NR³, S(O)ₘA and/or carbonyl oxygen,
Hal is F, Cl, Br or I,
m and
n are each, independently of one another, 0, 1 or 2,
and pharmaceutically usable derivatives, solvates and stereoisomers thereof, including mixtures thereof in all ratios.

2. Compounds according to claim 1, in which
R² is H,
and pharmaceutically usable derivatives, solvates and stereoisomers thereof, including mixtures thereof in all ratios.

3. Compounds according to claim 1 or 2, in which
R¹ is —C(═NH)—NH₂, which is unsubstituted or monosubstituted by OH, or is 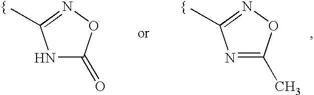
and pharmaceutically usable derivatives, solvates and stereoisomers thereof, including mixtures thereof in all ratios.

4. Compounds according to claim 1, in which
R¹ is CONH₂, CH₂NH₂, or —C(═NH)—NH₂, which is unsubstituted or monosubstituted by OH, and
R² is H,
and pharmaceutically usable derivatives, solvates and stereoisomers thereof, including mixtures thereof in all ratios.

5. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH, and
R^{2′} is H,
and pharmaceutically usable derivatives, solvates and stereoisomers thereof, including mixtures thereof in all ratios.

6. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H, and
R³ is H, A or —(CH₂)ₙ—Ar,
and pharmaceutically usable derivatives, solvates and stereoisomers thereof, including mixtures thereof in all ratios.

7. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H, and
R³ is H, alkyl having 1-6 carbon atoms, phenyl or benzyl,
and pharmaceutically usable derivatives, solvates and stereoisomers thereof, including mixtures thereof in all ratios.

8. Compounds according to claims 1-7, in which
Ar is phenyl, which is unsubstituted or monosubstituted or disubstituted by Hal, OR⁴, SO₂NH₂, SO₂A or NHCONH₂,
and pharmaceutically usable derivatives, solvates and stereoisomers thereof, including mixtures thereof in all ratios.

9. Compounds according to claims 1-8, in which
X is CONR³, CH₂CONR³, CH₂NR³, CONR³CH₂, CH₂O, CH₂OCH₂ or OCH₂,
R³ is H, alkyl having 1, 2, 3, 4, 5 or 6 carbon atoms, phenyl or benzyl,
and pharmaceutically usable derivatives, solvates and stereoisomers thereof, including mixtures thereof in all ratios.

10. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H,
X is CONR³, CH₂CONR³, CH₂NR³, CONR³CH₂, CH₂O, CH₂OCH₂ or OCH₂,
R³ is H, alkyl having 1, 2, 3, 4, 5 or 6 carbon atoms, phenyl or benzyl,
and pharmaceutically usable derivatives, solvates and stereoisomers thereof, including mixtures thereof in all ratios.

11. Compounds according to claim 1, in which
R¹ is CONH₂, CH₂NH₂, or —C(═NH)—NH₂, which is unsubstituted or monosubstituted by OH,
R² is H,
R² is H, Hal, A, ═CH—COOA, ═CH—CONH₂ or O—CH₂—COOH,
R^{2″} is H,
X is CONH, CONHCH₂, CH₂NH or CH₂O,
R³ is H, alkyl having 1, 2, 3, 4, 5 or 6 carbon atoms, phenyl or benzyl,
and pharmaceutically usable derivatives, solvates and stereoisomers thereof, including mixtures thereof in all ratios.

12. Compounds according to claims 1-11, in which
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
and pharmaceutically usable derivatives, solvates and stereoisomers thereof, including mixtures thereof in all ratios.

13. Compounds according to claims 1-12, in which
Y is Ar-diyl,
and pharmaceutically usable derivatives, solvates and stereoisomers thereof, including mixtures thereof in all ratios.

14. Compounds according to claims 1-13, in which
Y is Ar-diyl, and
Ar is phenyl, which is unsubstituted or monosubstituted or disubstituted by Hal, OR⁴, SO₂NH₂, SO₂A or NHCONH₂,
and pharmaceutically usable derivatives, solvates and stereoisomers thereof, including mixtures thereof in all ratios.

15. Compounds according to claims 1-14, in which
Y is 1,4-phenylene,
and pharmaceutically usable derivatives, solvates and stereoisomers thereof, including mixtures thereof in all ratios.

16. Compounds according to claims 1-15, in which
T is a monocyclic or bicyclic, saturated or unsaturated heterocyclic ring having 1 or 2 N and/or O atoms, which is monosubstituted or disubstituted by carbonyl oxygen,
and pharmaceutically usable derivatives, solvates and stereoisomers thereof, including mixtures thereof in all ratios.

17. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
X is CONR³, CH₂CONR³, CH₂NR³, CONR³CH₂, CH₂O, CH₂OCH₂ or OCH₂,
and pharmaceutically usable derivatives, solvates and stereoisomers thereof, including mixtures thereof in all ratios.

18. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
X is CONR³, CH₂CONR³, CH₂NR³, CONR³CH₂, CH₂O, CH₂OCH₂ or OCH₂,
Y is Ar-diyl, and
Ar is phenyl,
and pharmaceutically usable derivatives, solvates and stereoisomers thereof, including mixtures thereof in all ratios.

19. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
X is CONR³, CH₂CONR³, CH₂NR³, CONR³CH₂, CH₂O, CH₂OCH₂ or OCH₂,
Y is Ar-diyl,
Ar is phenyl, which is unsubstituted or monosubstituted or disubstituted by Hal, OR⁴, SO₂NH₂, SO₂A or NHCONH₂,
T is a monocyclic or bicyclic, saturated or unsaturated heterocyclic ring having 1 or 2 N and/or O atoms, which is monosubstituted or disubstituted by carbonyl oxygen,
and pharmaceutically usable derivatives, solvates and stereoisomers thereof, including mixtures thereof in all ratios.

20. Compounds according to claim 1, in which
R¹ is CONH₂, CH₂NH₂, or —C(═NH)—NH₂, which is unsubstituted or monosubstituted by OH,
R² is H,
R² is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
X is CONR³, CH₂CONR³, CH₂NR³, CONR³CH₂, CH₂O, CH₂OCH₂ or OCH₂,
Y is Ar-diyl,
Ar is phenyl, which is unsubstituted or monosubstituted or disubstituted by Hal, OR⁴, SO₂NH₂, SO₂A or NHCONH₂,
T is 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2,6-dioxopiperidin-1-yl, 2-oxopiperazin-1-yl, 2,5-dioxopyrrolidin-1-yl, 2-oxo-1,3-oxazolidin-3-yl or 3-oxo-2H-pyridazin-2-yl,
and pharmaceutically usable derivatives, solvates and stereoisomers thereof, including mixtures thereof in all ratios.

21. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
X is CONR³, CH₂CONR³, CH₂NR³, CONR³CH₂, CH₂O, CH₂OCH₂ or OCH₂,
Y is 1,4-phenylene,
T is 2-oxopiperidin-1-yl, 2-oxopyrrolidin-1-yl, 2-oxo-1H-pyridin-1-yl, 3-oxomorpholin-4-yl, 4-oxo-1 H-pyridin-1-yl, 2,6-dioxopiperidin-1-yl, 2-oxopiperazin-1-yl, 2,5-dioxopyrrolidin-1-yl, 2-oxo-1,3-oxazolidin-3-yl or 3-oxo-2H-pyridazin-2-yl,
and pharmaceutically usable derivatives, solvates and stereoisomers thereof, including mixtures thereof in all ratios.

22. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
X is CONH, CONHCH₂, CH₂NH or CH₂O,
Y is 1,4-phenylene,
T is 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2,6-dioxopiperidin-1-yl, 2-oxopiperazin-1-yl, 2,5-dioxopyrrolidin-1-yl, 2-oxo-1,3-oxazolidin-3-yl or 3-oxo-2H-pyridazin-2-yl,
and pharmaceutically usable derivatives, solvates and stereoisomers thereof, including mixtures thereof in all ratios.

23. Compounds according to claim 1, in which
R¹ is CONH₂, CH₂NH₂, or —C(═NH)—NH₂, which is unsubstituted or monosubstituted by OH,
R² is H,
R² is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
X is CONH, CONHCH₂, CH₂NH or CH₂O,
Y is 1,4-phenylene,
T is 2-oxopiperidin-1-yl, 2-oxopyrrolidin-1-yl, 2-oxo-1H-pyridin-1-yl, 4-oxo-1H-pyridin-1-yl, 2-oxopiperazin-1-yl, 2- or 3-oxo-2H-pyridazin-2-yl,
and pharmaceutically usable derivatives, solvates and stereoisomers thereof, including mixtures thereof in all ratios.

24. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
X is CONH, CONHCH₂, CH₂NH or CH₂O,
Y is 1,4-phenylene,
T is 2-oxopiperidin-1-yl, 2-oxopyrrolidin-1-yl, 2-oxo-1H-pyridin-1-yl, 4-oxo-1H-pyridin-1-yl, 2-oxopiperazin-1-yl, 2- or 3-oxo-2H-pyridazin-2-yl or 2-aza-bicyclo[2.2.2]octan-3-on-2-yl,
A is unbranched or branched alkyl having 1-6 carbon atoms, and in addition 1-7H atoms may be replaced by F,
Hal is F, Cl or Br,
and pharmaceutically usable derivatives, solvates and stereoisomers thereof, including mixtures thereof in all ratios.

25. Compounds according to claim 1, in which
R¹ is CONH₂, CH₂NH₂, or —C(═NH)—NH₂, which is unsubstituted or monosubstituted by OH,
R² is H,
R^{2′} is H, Hal, A, ═CH—COOA, ═CH—CONH₂ or O—CH₂—COOH,
R^{2″} is H,
R³ is H, alkyl having 1, 2, 3, 4, 5 or 6 carbon atoms, phenyl or benzyl,
W is N or CH or an sp²-hybridised carbon atom,
E together with W is a 3- to 7-membered saturated carbocyclic or heterocyclic ring having from 0 to 2 N atoms which
a) may contain a double bond
and to which
b) may be fused a benzene ring or a saturated or aromatic heterocyclic ring having 1-2 N atoms,
which
c) may be substituted by carbonyl oxygen,
X is CONH, CONHCH₂, CH₂NH or CH₂O,
Y is 1,4-phenylene,
T is 2-oxopiperidin-1-yl, 2-oxopyrrolidin-1-yl, 2-oxo-1H-pyridin-1-yl, 4-oxo-1H-pyridin-1-yl, 2-oxopiperazin-1-yl, 2- or 3-oxo-2H-pyridazin-2-yl or 2-aza-bicyclo[2.2.2]octan-3-on-2-yl,
A is unbranched or branched alkyl having 1-6 carbon atoms, and in addition 1-7H atoms may be replaced by F,
Hal is F, Cl or Br,
and pharmaceutically usable derivatives, solvates and stereoisomers thereof, including mixtures thereof in all ratios.

26. Compounds according to claim 1, selected from the group consisting of
tert-butyl 4-(3-carbamoylphenyl)-3-[4-(2-oxopiperidin-1-yl)phenylcarbamoyl]piperazine-1-carboxylate,
N-[4-(2-oxopiperidin-1-yl)phenyl]-1-(3-carbamoylphenyl)piperazine-2-carboxamide,
N-[4-(2-oxopiperidin-1-yl)phenyl]-1-(3-carbamoylphenyl)piperidine-2-carboxamide,
N-[4-(2-oxopiperidin-1-yl)phenyl]-1-(3-carbamoylphenyl)pyrrolidine-2-carboxamide, N-[4-(2-oxopiperidin-1-yl)phenyl]-1-(3-carbamoylphenyl)-2,3-dihydro-1H-isoindole-1-carboxamide,
N-[4-(2-oxopiperazin-1-yl)phenyl]-1-(3-carbamoylphenyl)piperidine-2-carboxamide,
N-[4-(2-oxopyridin-1-yl)phenyl]-1-(3-carbamoylphenyl)piperidine-2-carboxamide,
N-[4-(2-oxopiperidin-1-yl)phenyl]-1-(3-carbamoylphenyl)-4-(2,2,2-trifluoroethyl)piperidinecarboxamide,
N-[4-(2-oxopiperidin-1-yl)phenylmethyl]-1-(3-carbamoylphenyl)-piperidine-2-carboxamide,
N-[4-(2-oxopiperidin-1-yl)phenyl]-2-(3-cyanophenyl)cyclopent-1-enecarboxamide,
N-[4-(2-oxopiperidin-1-yl)phenyl]-2-[3-(N-hydroxyamidino)phenyl]-cyclopent-1-enecarboxamide,
N-[4-(2-oxopiperidin-1-yl)phenyl]-2-(3-amidinophenyl)cyclopent-1-enecarboxamide,
3-{2-[4-(2-oxopiperidin-1-yl)phenylcarbamoyl]cyclopent-1-enyl}-benzamide,
3-{cis-2-[4-(2-oxopiperidin-1-yl)phenylcarbamoyl]cyclopentyl}-benzamide,
N-[4-(2-oxopiperidin-1-yl)phenyl]-cis-2-(3-aminomethylphenyl)-cyclopentanecarboxamide,
N-[4-(2-oxopiperidin-1-yl)phenyl]-cis-2-(3-aminomethylphenyl)2-cyclopentanecarboxamide,
N-[4-(2-oxopiperidin-1-yl)phenyl]-cis-2-(3-aminomethylphenyl)-cyclopropanecarboxamide,
N-[4-(2-oxopiperidin-1-yl)phenyl]-2-(3-amidinophenyl)piperidine-2-carboxamide,
N-[4-(2-oxopiperidin-1-yl)phenyl]-2-(3-aminomethylphenyl)-piperidine-2-carboxamide,
3-{2-[4-(2-oxopiperidin-1-yl)phenylcarbamoyl]cyclohex-1-enyl}-benzamide,
3-{2-[4-(2-oxopiperidin-1-yl)phenylcarbamoyl]cyclohexyl}benzamide,
N-[4-(2-oxopiperidin-1-yl)phenyl]-4-(3-carbamoylphenyl)-1,2,5,6-tetrahydropyridine-3-carboxamide,
3-{2-[4-(2-oxopiperidin-1-yl)phenylcarbamoyl]cyclopentyl}benzamide,
N-[4-(2-oxopiperidin-1-yl)phenyl]-2-(3-aminomethylphenyl)cyclo-pentanecarboxamide,
3-(2-{[4-(2-oxopiperidin-1-yl)phenylamino]methyl}piperidin-1-yl)-benzamide,
3-(2-{[4-(2-oxopiperidin-1-yl)phenoxy]methyl}piperidin-1-yl)benzamide,
{1-(3-carbamoylphenyl)-6-[4-(2-oxopiperidin-1-yl)phenylcarbamoyl]-piperidin-3-ylidene}acetamide,
{1-(3-carbamoylphenyl)-6-[4-(2-oxopiperidin-1-yl)phenylcarbamoyl]-piperidin-3-yloxy}acetic acid,
5-(3-aminomethylphenyl)-1-methyl-4,5,6,7-tetrahydro-1H—N-[4-(2-oxopiperidin-1-yl)phenyl]-imidazo[4,5-c]pyridine-6-carboxamide,
{5-(3-carbamoylphenyl)-6-[4-(2-oxopiperidin-1-yl)phenylcarbamoyl]-4,5,6,7-tetrahydroimidazo[4,5-c]pyridin-1-yl}acetamide,
N-[4-(2-oxopiperidin-1-yl)-2-fluorophenyl]-2-(3-aminomethylphenyl)-piperidine-2-carboxamide,
N-[4-(2-oxopiperidin-1-yl)phenyl]-(S)-2-(3-aminomethylphenyl)-5-oxopyrrolidine-2-carboxamide,
N-[4-(2-oxopiperidin-1-yl)phenyl]-(R)-2-(3-aminomethylphenyl)-pyrrolidine-2-carboxamide,
N-[4-(3-oxo-2-azabicyclo[2.2.2]oct-2-yl)phenyl]-2-(3-amidinophenyl)-piperidine-2-carboxamide,
N-[4-(3-oxo-2-azabicyclo[2.2.2]oct-2-yl)phenyl]-2-[3-(N-hydroxyamidino)phenyl]piperidine-2-carboxamide,
and pharmaceutically usable derivatives, solvates and stereoisomers thereof, including mixtures thereof in all ratios.

27. Process for the preparation of compounds of the formula I according to claims 1-25 and pharmaceutically tolerated salts and solvates thereof, characterised in that
a) they are liberated from one of their functional derivatives by treatment with a solvolysing or hydrogenolysing agent by
i) liberating an amidino group from their hydroxyl, oxadiazole or oxazolidinone derivative by hydrogenolysis or solvolysis,
ii) replacing a conventional amino-protecting group with hydrogen by treatment with a solvolysing or hydrogenolysing agent, or liberating an amino group protected by a conventional protecting group, or
b) a cyano group is converted into an N-hydroxyamidino group, or
c) for the preparation of a compound of the formula I in which X is —[C(R⁴)₂]ₙCONR³[C(R⁴)₂]ₙ—, —[C(R⁴)₂]ₙNR³[C(R⁴)₂]ₙ—or —[C(R⁴)₂]ₙO[C(R⁴)₂]ₙ—,
a compound of the formula II 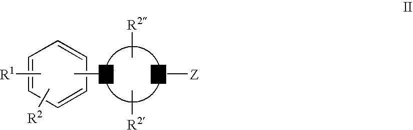
in which
Z is —[C(R⁴)₂]ₙCO-L or —[C(R⁴)₂]ₙ-L,
L is Cl, Br, I or a free or reactively functionally modified OH group, and
R¹, R², R^{2′}, R², R⁴, n, W and E are as defined in claim 1
with the proviso that any free amino group present is protected,
is reacted with a compound of the formula III
Q-Y-T III
in which
Q is HNR³[C(R⁴)₂]ₙ-Y-T or HO[C(R⁴)₂]ₙ—Y-T,
and R³, R⁴, n, Y and T are as defined in claim 1,
and, where appropriate, a protecting group is subsequently removed or
d) for the preparation of a compound of the formula I
in which X is —[C(R⁴)₂]ₙNR³CO[C(R⁴)₂]ₙ—,
a compound of the formula IV 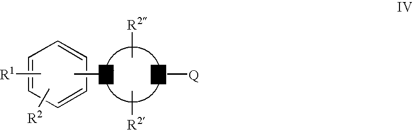
in which
Q is —[C(R⁴)₂]ₙNHR³,
and R¹, R², R^{2′}, R^{2″}, R³, R⁴, n, W and E are as defined in claim 1, with the proviso that any further free amino group present is protected,
is reacted with a compound of the formula V
Z-Y-T V
in which
Z is L-C(═OY[C(R⁴)₂]ₙ-Y-T, and
L is Cl, Br, I or a free or reactively functionally modified OH group, and
n, Y and T are as defined in claim 1,
and, where appropriate, a protecting group, is subsequently removed and/or
e) a base or acid of the formula I is converted into one of its salts.

28. Compounds of the formula I according to one or more of claims 1 to 26 as inhibitors of coagulation factor Xa.

29. Compounds of the formula I according to one or more of claims 1 to 26 as inhibitors of coagulation factor VIIa.

30. Medicament comprising at least one compound of the formula I according to one or more of claims 1 to 26 and/or pharmaceutically usable derivatives, solvates and stereoisomers thereof, including mixtures thereof in all ratios, and, if desired, excipients and/or assistants.

31. Medicament comprising at least one compound of the formula I according to one or more of claims 1 to 26 and/or pharmaceutically usable derivatives, solvates and stereoisomers thereof, including mixtures thereof in all ratios, and at least one further medicament active ingredient.

32. Use of compounds according to one or more of claims 1 to 26 and/or physiologically acceptable salts and solvates thereof for the preparation of a medicament for the treatment of thromboses, myocardial infarction, arteriosclerosis, inflammation, apoplexia, angina pectoris, restenosis after angioplasty, claudicatio intermittens, tumours, tumour diseases and/or tumour metastases.

33. Set (kit) consisting of separate packs of
(a) an effective amount of a compound of the formula I according to one or more of claims 1 to 26 and/or pharmaceutically usable derivatives, solvates and stereoisomers thereof, including mixtures thereof in all ratios, and
(b) an effective amount of a further medicament active ingredient.

34. Use of compounds of the formula I according to one or more of claims 1 to 26 and/or pharmaceutically usable derivatives, solvates and stereoisomers thereof, including mixtures thereof in all ratios, for the preparation of a medicament for the treatment of thromboses, mycocardial infarction, arteriosclerosis, inflammation, apoplexia, angina pectoris, restenosis after angioplasty, claudicatio intermittens, tumours, tumour diseases and/or tumour metastases, in combination with at least one further medicament active ingredient.
